SEQUENCE LISTING (92 sequences)

SEQ ID NO: 1 - DNA, 4361 bp, Homo sapiens
  source 1..4361: mol_type = genomic DNA
aggctgctggagaaggcgcacctgctgcaggtgctcccggccgccccggaccagcgagcgcgggcactgcggcggggaggatgctgcgcgagcggaccgtgcggctgcagtacgggagccgcgtggaggcggtgtacgtgctgggcacctacctctggaccgatgtctacagcgcggccccagccggggcccaaaccttcagcctgaagcactcggaacacgtgtgggtggaggtggtgcgtgatggggaggctgaggaggtggccaccaatggcaagcagcgctggcttctctcgcccagcaccaccctgcgggtcaccatgagccaggcgagcaccgaggccagcagtgacaaggtcaccgtcaactactatgacgaggaagggagcattcccatcgaccaggcggggctcttcctcacagccattgagatctccctggatgtggacgcagaccgggatggtgtggtggagaagaacaacccaaagaaggcatcctggacctggggccccgagggccagggggccatcctgctggtgaactgtgaccgagagacaccctggttgcccaaggaggactgccgtgatgagaaggtctacagcaaggaagatctcaaggacatgtcccagatgatcctgcggaccaaaggccccgaccgcctccccgccggatacgagatagttctgtacatttccatgtcagactcagacaaagtgggcgtgttctacgtggagaacccgttcttcggccaacgctatatccacatcctgggccggcggaagctctaccatgtggtcaagtacacgggtggctccgcggagctgctgttcttcgtggaaggcctctgtttccccgacgagggcttctcaggcctggtctccatccatgtcagcctgctggagtacatggcccaggacattcccctgactcccatcttcacggacaccgtgatattccggattgctccgtggatcatgacccccaacatcctgcctcccgtgtcggtgtttgtgtgctgcatgaaggataattacctgttcctgaaagaggtgaagaaccttgtggagaaaaccaactgtgagctgaaggtctgcttccagtacctaaaccgaggcgatcgctggatccaggatgaaattgagtttggctacatcgaggccccccataaaggcttccccgtggtgctggactctccccgagatggaaacctaaaggacttccctgtgaaggagctcctgggcccagattttggctacgtgacccgggagcccctctttgagtctgtcaccagccttgactcatttggaaacctggaggtcagtcccccagtgaccgtgaacggcaagacatacccgcttggccgcatcctcatcgggagcagctttcctctgtctggtggtcggaggatgaccaaggtggtgcgtgacttcctgaaggcccagcaggtgcaggcgcccgtggagctctactcagactggctgactgtgggccacgtggatgagttcatgtcctttgtccccatccccggcacaaagaaattcctgctactcatggccagcacctcggcctgctacaagctcttccgagagaagcagaaggacggccatggagaggccatcatgttcaaaggcttgggtgggatgagcagcaagcgaatcaccatcaacaagattctgtccaacgagagccttgtgcaggagaacctgtacttccagcgctgcctagactggaaccgtgacatcctcaagaaggagctgggactgacagagcaggacatcattgacctgcccgctctgttcaagatggacgaggaccaccgtgccagagccttcttcccaaacatggtgaacatgatcgtgctggacaaggacctgggcatccccaagccattcgggccacaggttgaggaggaatgctgcctggagatgcacgtgcgtggcctcctggagcccctgggcctcgaatgcaccttcatcgacgacatttctgcctaccacaaatttctgggggaagtccactgtggcaccaacgtccgcaggaagcccttcaccttcaagtggtggcacatggtgccctgacctgccaggggccctggcgtttgcctccttcgcttagttctccagaccctccctcacacgcccagagccttctgctgacatggactggacagccccgctgggagacctttgggacgtggggtggaatttggggtatctgtgccttgccctccctgagaggggcctcagtgtcctctgaagccatccccagtgagcctcgactctgtccctgctgaaaatagctgggccagtgtctctgtagccctgacataaggaacagaacacaacaaaacacagcaaaccatgtgcccaaactgctccccaaagaattttgagtctctaatctgacactgaatgaggggagaagggaaggagattctgggattgccagttcttccagcagccatgctctgaaaatcaaggtagaatccatggaaagggaccccaggaccccgggaccctagacgtatcttgaactgccatcgtcatttcaaatacatctccctcagggtttccaggtggccacccccaattattcattccttaccaacctctcaaatcctcttggctttctctctgcagtgtggacactgttggctagtcctccccactccctgagggtccagtaagttagcttagaaccttcctggaaacatttcatctgagcaggtttccccacgtgtgggatgctccttttgcctcatctgtctcagggatgcaggctcccccgcatgcatggggatttctccccagaccagcatacttgtgacctgagagttcaatgcgtaaagatgcccctggtcagccatatccatcttctcttgcctggtccttgattctctggccgctccctgaccttcctccttccactgccttgactttcttcctttttattcctggtgccatctgtccaggcagctagacaagaacttgttcgccagcagccagattcaggccttcccaggggcataataagtgaccagcccctcctctccggacatcagatccaacacataaggaccctggcctaccctccagcccaacagccagttctgggtcagctgccaacttaggggtggtttgattatcccattgaaattcaccagtgcctttgccaaagaccctctcatttggacatacccagattcattccctggctccaactgaaaagactcagtttcaatcgttaaaagttcctttagggccagaagaataaatgaattataatcccattttgaagaaccgatttataaccaatgaaaaggttataatgtaatttatattcttggaggaacaagattttcatttgggattatttccttcaaccattcaacaaacatttgttgtatgccactaagcgccaggcacggcgttgggctctgcaaacacagtggttagtagcagtctggacctggtccctactggcatggaacccatcactccccaacatgcaaagcccacatttaaaggccagcctctgccccttcagtgatgcgctctttagaaatgccagtccactatattcagaaatccgcagggcacaaaacttccagcaagtcactgttgtggtgaaatgggcagtgggggtggggggtcttctttaaacaggcccccttcccatctacctagccagtacccatccaatgagtccccagagcctccagaagctgttgtctcctctctggggacagcagctcctgcctttggaggccaaagccccagatctctccagccccagagctgaaaacaccaagtgcctatttgagggtgtctgtctggagacttagagtttgtcatgtgtgtgtgtgtgtttggttaatgtgggtttatgggttttctttcttttttttctttttttttttagtctacattagggggaagtgagcgcctcccatgtgcagacagtgtgtctttatagatttttctaaggctttccccaatgatgtcggtaatttctgatgtttctgaagttcccaggactcacacacccgttcccatctcacttgcccacccagtgtgacaaccctcggtgtggatatacccccgtggactcatggctcttccccacccccactttctataaatgtaggcctagaatacgcttctctgttgcaaaactcagctaagttcctgcttccaccttgatgttgaaatatcttatgtaagagggcaggggatgtcgtgaagatggcaagaagaacacagtttcaaatttctggaaaagagcctgtggtggagatctaaagatgtttagggaagagctcgactaaagaacaatgaaataaatggtccaaggggaagtca

SEQ ID NO: 2 - protein, 665 aa, Homo sapiens
MLRERTVRLQYGSRVEAVYVLGTYLWTDVYSAAPAGAQTFSLKHSEHVWVEVVRDGEAEEVATNGKQRWLLSPSTTLRVTMSQASTEASSDKVTVNYYDEEGSIPIDQAGLFLTAIEISLDVDADRDGVVEKNNPKKASWTWGPEGQGAILLVNCDRETPWLPKEDCRDEKVYSKEDLKDMSQMILRTKGPDRLPAGYEIVLYISMSDSDKVGVFYVENPFFGQRYIHILGRRKLYHVVKYTGGSAELLFFVEGLCFPDEGFSGLVSIHVSLLEYMAQDIPLTPIFTDTVIFRIAPWIMTPNILPPVSVFVCCMKDNYLFLKEVKNLVEKTNCELKVCFQYLNRGDRWIQDEIEFGYIEAPHKGFPVVLDSPRDGNLKDFPVKELLGPDFGYVTREPLFESVTSLDSFGNLEVSPPVTVNGKTYPLGRILIGSSFPLSGGRRMTKVVRDFLKAQQVQAPVELYSDWLTVGHVDEFMSFVPIPGTKKFLLLMASTSACYKLFREKQKDGHGEAIMFKGLGGMSSKRITINKILSNESLVQENLYFQRCLDWNRDILKKELGLTEQDIIDLPALFKMDEDHRARAFFPNMVNMIVLDKDLGIPKPFGPQVEEECCLEMHVRGLLEPLGLECTFIDDISAYHKFLGEVHCGTNVRRKPFTFKWWHMVP

SEQ ID NO: 3 - DNA, 3459 bp, Homo sapiens
  source 1..3459: mol_type = genomic DNA
agcagctctgcagatggggattcttctgtcagctcatatctgcacatgtgcacaaagacataaacatacaagtgcactacagtgctgcttcttgtagtagcaaaagattgcaaagcaacctaaatgtccatctgtggaggactgagtaacggccttcccggaagttctgtgcaaccgctaaaaaggatgaagccagtctctcaatatggatacaggatgtgcttcaaggatgaaattgagtttggctacatcgaggccccccataaaggcttccccgtggtgctggactctccccgagatggaaacctaaaggacttccctgtgaaggagctcctgggcccagattttggctacgtgacccgggagcccctctttgagtctgtcaccagccttgactcatttggaaacctggaggtcagtcccccagtgaccgtgaacggcaagacatacccgcttggccgcatcctcatcgggagcagctttcctctgtctggtggtcggaggatgaccaaggtggtgcgtgacttcctgaaggcccagcaggtgcaggcgcccgtggagctctactcagactggctgactgtgggccacgtggatgagttcatgtcctttgtccccatccccggcacaaagaaattcctgctactcatggccagcacctcggcctgctacaagctcttccgagagaagcagaaggacggccatggagaggccatcatgttcaaaggcttgggtgggatgagcagcaagcgaatcaccatcaacaagattctgtccaacgagagccttgtgcaggagaacctgtacttccagcgctgcctagactggaaccgtgacatcctcaagaaggagctgggactgacagagcaggacatcattgacctgcccgctctgttcaagatggacgaggaccaccgtgccagagccttcttcccaaacatggtgaacatgatcgtgctggacaaggacctgggcatccccaagccattcgggccacaggttgaggaggaatgctgcctggagatgcacgtgcgtggcctcctggagcccctgggcctcgaatgcaccttcatcgacgacatttctgcctaccacaaatttctgggggaagtccactgtggcaccaacgtccgcaggaagcccttcaccttcaagtggtggcacatggtgccctgacctgccaggggccctggcgtttgcctccttcgcttagttctccagaccctccctcacacgcccagagccttctgctgacatggactggacagccccgctgggagacctttgggacgtggggtggaatttggggtatctgtgccttgccctccctgagaggggcctcagtgtcctctgaagccatccccagtgagcctcgactctgtccctgctgaaaatagctgggccagtgtctctgtagccctgacataaggaacagaacacaacaaaacacagcaaaccatgtgcccaaactgctccccaaagaattttgagtctctaatctgacactgaatgaggggagaagggaaggagattctgggattgccagttcttccagcagccatgctctgaaaatcaaggtagaatccatggaaagggaccccaggaccccgggaccctagacgtatcttgaactgccatcgtcatttcaaatacatctccctcagggtttccaggtggccacccccaattattcattccttaccaacctctcaaatcctcttggctttctctctgcagtgtggacactgttggctagtcctccccactccctgagggtccagtaagttagcttagaaccttcctggaaacatttcatctgagcaggtttccccacgtgtgggatgctccttttgcctcatctgtctcagggatgcaggctcccccgcatgcatggggatttctccccagaccagcatacttgtgacctgagagttcaatgcgtaaagatgcccctggtcagccatatccatcttctcttgcctggtccttgattctctggccgctccctgaccttcctccttccactgccttgactttcttcctttttattcctggtgccatctgtccaggcagctagacaagaacttgttcgccagcagccagattcaggccttcccaggggcataataagtgaccagcccctcctctccggacatcagatccaacacataaggaccctggcctaccctccagcccaacagccagttctgggtcagctgccaacttaggggtggtttgattatcccattgaaattcaccagtgcctttgccaaagaccctctcatttggacatacccagattcattccctggctccaactgaaaagactcagtttcaatcgttaaaagttcctttagggccagaagaataaatgaattataatcccattttgaagaaccgatttataaccaatgaaaaggttataatgtaatttatattcttggaggaacaagattttcatttgggattatttccttcaaccattcaacaaacatttgttgtatgccactaagcgccaggcacggcgttgggctctgcaaacacagtggttagtagcagtctggacctggtccctactggcatggaacccatcactccccaacatgcaaagcccacatttaaaggccagcctctgccccttcagtgatgcgctctttagaaatgccagtccactatattcagaaatccgcagggcacaaaacttccagcaagtcactgttgtggtgaaatgggcagtgggggtggggggtcttctttaaacaggcccccttcccatctacctagccagtacccatccaatgagtccccagagcctccagaagctgttgtctcctctctggggacagcagctcctgcctttggaggccaaagccccagatctctccagccccagagctgaaaacaccaagtgcctatttgagggtgtctgtctggagacttagagtttgtcatgtgtgtgtgtgtgtttggttaatgtgggtttatgggttttctttcttttttttctttttttttttagtctacattagggggaagtgagcgcctcccatgtgcagacagtgtgtctttatagatttttctaaggctttccccaatgatgtcggtaatttctgatgtttctgaagttcccaggactcacacacccgttcccatctcacttgcccacccagtgtgacaaccctcggtgtggatatacccccgtggactcatggctcttccccacccccactttctataaatgtaggcctagaatacgcttctctgttgcaaaactcagctaagttcctgcttccaccttgatgttgaaatatcttatgtaagagggcaggggatgtcgtgaagatggcaagaagaacacagtttcaaatttctggaaaagagcctgtggtggagatctaaagatgtttagggaagagctcgactaaagaacaatgaaataaatggtccaaggggaagtca

SEQ ID NO: 4 - protein, 350 aa, Homo sapiens
MSICGGLSNGLPGSSVQPLKRMKPVSQYGYRMCFKDEIEFGYIEAPHKGFPVVLDSPRDGNLKDFPVKELLGPDFGYVTREPLFESVTSLDSFGNLEVSPPVTVNGKTYPLGRILIGSSFPLSGGRRMTKVVRDFLKAQQVQAPVELYSDWLTVGHVDEFMSFVPIPGTKKFLLLMASTSACYKLFREKQKDGHGEAIMFKGLGGMSSKRITINKILSNESLVQENLYFQRCLDWNRDILKKELGLTEQDIIDLPALFKMDEDHRARAFFPNMVNMIVLDKDLGIPKPFGPQVEEECCLEMHVRGLLEPLGLECTFIDDISAYHKFLGEVHCGTNVRRKPFTFKWWHMVP

SEQ ID NO: 5 - DNA, 3189 bp, Homo sapiens
  source 1..3189: mol_type = genomic DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 6 - protein, 664 aa, Homo sapiens
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 7 - DNA, 1995 bp, Homo sapiens
  misc_feature 1..1995: note = Homo sapiens peptidyl arginine deiminase 3 (PADI3) transcript, mRNA 154A>G
  source 1..1995: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacgtctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 8 - protein, 664 aa, synthetic construct
  REGION 1..664: note = source = /note="Description of Artificial Sequence: Synthetic polypeptide"
MSLQRIVRVSLEHPTSAVCVAGVETLVDIYGSVPEGTEMFEVYGTPGVDIYVSPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 9 - DNA, 1995 bp, Homo sapiens
  misc_feature 1..1995: note = Homo sapiens peptidyl arginine deiminase 3 (PADI3) transcript, mRNA 335T>A
  source 1..1995: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctaccacacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 10 - protein, 664 aa, synthetic construct
  REGION 1..664: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYHTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 11 - DNA, 1995 bp, Homo sapiens
  misc_feature 1..1995: note = Homo sapiens peptidyl arginine deiminase 3 (PADI3) transcript, mRNA 511G>A
  source 1..1995: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacatgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 12 - protein, 664 aa, synthetic construct
  REGION 1..664: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHMHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 13 - DNA, 1995 bp, Homo sapiens
  misc_feature 1..1995: note = Homo sapiens peptidyl arginine deiminase 3 (PADI3) transcript, mRNA 811C>T
  source 1..1995: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 14 - protein, 664 aa, synthetic construct
  REGION 1..664: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 15 - DNA, 1995 bp, Homo sapiens
  misc_feature 1..1995: note = Homo sapiens peptidyl arginine deiminase 3 (PADI3) transcript, mRNA 1744G>A
  source 1..1995: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacgaccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 16 - protein, 664 aa, synthetic construct
  REGION 1..664: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATTFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 17 - DNA, 1995 bp, Homo sapiens
  misc_feature 1..1995: note = Homo sapiens peptidyl arginine deiminase 3 (PADI3) transcript, mRNA 1813C>A
  source 1..1995: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggaccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 18 - protein, 664 aa, synthetic construct
  REGION 1..664: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGTIINGCCCLEEKVRSLLEPLGLHCTFIDDFTPYHMLHGEVHCGTNVCRKPFSFKWWNMVP

SEQ ID NO: 19 - DNA, 1995 bp, Homo sapiens
  misc_feature 1..1995: note = Homo sapiens peptidyl arginine deiminase 3 (PADI3) transcript, mRNA 1853G>A
  source 1..1995: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcagtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 20 - protein, 664 aa, synthetic construct
  REGION 1..664: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVQSLLEPLGLHCTFIDDFTPYHMLHGEVHCGTNVCRKPFSFKWWNMVP

SEQ ID NO: 21 - DNA, 4800 bp, Homo sapiens
  source 1..4800: mol_type = genomic DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 22 - protein, 626 aa, Homo sapiens
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 23 - DNA, 1881 bp, Homo sapiens
  misc_feature 1..1881: note = PREDICTED: Homo sapiens peptidyl arginine deiminase 3 (PADI3), transcript variant X1, mRNA 40A>G
  source 1..1881: mol_type = genomic DNA
atgtttgaggtctatgggacgcctggcgtggacatctacgtctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 24 - protein, 626 aa, synthetic construct [sequence omitted]

SEQ ID NO: 25 - DNA, 1881 bp, Homo sapiens
  misc_feature 1..1881: note = PREDICTED: Homo sapiens peptidyl arginine deiminase 3 (PADI3), transcript variant X1, mRNA 221T>A
  source 1..1881: mol_type = genomic DNA
atgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctaccacacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 26 - protein, 626 aa, synthetic construct
  REGION 1..626: note = source = /note="Description of Artificial Sequence: Synthetic polypeptide"
MFEVYGTPGVDIYISPNMERGRERADTRRWRFDATLEIIVVMNSPSNDLNDSHVQISYHSSHEPLPLAYAVLYHTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 27 - DNA, 1881 bp, Homo sapiens
  misc_feature 1..1881: note = PREDICTED: Homo sapiens peptidyl arginine deiminase 3 (PADI3), transcript variant X1, mRNA 397G>A
  source 1..1881: mol_type = genomic DNA
atgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacatgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 28 - protein, 626 aa, synthetic construct
  REGION 1..626: note = source = /note="Description of Artificial Sequence: Synthetic polypeptide"
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHMHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 29 - DNA, 1881 bp, Homo sapiens
  misc_feature 1..1881: note = PREDICTED: Homo sapiens peptidyl arginine deiminase 3 (PADI3), transcript variant X1, mRNA 767C>T
  source 1..1881: mol_type = genomic DNA
atgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 30 - protein, 626 aa, synthetic construct
  REGION 1..626: note = source = /note="Description of Artificial Sequence: Synthetic polypeptide"
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 31 - DNA, 1881 bp, Homo sapiens
  misc_feature 1..1881: note = PREDICTED: Homo sapiens peptidyl arginine deiminase 3 (PADI3), transcript variant X1, mRNA 1630G>A
  source 1..1881: mol_type = genomic DNA
atgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacgaccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 32 - protein, 626 aa, synthetic construct
  REGION 1..626: note = source = /note="Description of Artificial Sequence: Synthetic polypeptide"
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATTFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 33 - DNA, 1881 bp, Homo sapiens
  misc_feature 1..1881: note = PREDICTED: Homo sapiens peptidyl arginine deiminase 3 (PADI3), transcript variant X1, mRNA 1699C>A
  source 1..1881: mol_type = genomic DNA
atgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggaccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 34 - protein, 626 aa, synthetic construct
  REGION 1..626: note = source = /note="Description of Artificial Sequence: Synthetic polypeptide"
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGTIINGCCCLEEKVRSLLEPLGLHCTFIDDFTPYHMLHGEVHCGTNVCRKPFSFKWWNMVP

SEQ ID NO: 35 - DNA, 1881 bp, Homo sapiens
  misc_feature 1..1881: note = PREDICTED: Homo sapiens peptidyl arginine deiminase 3 (PADI3), transcript variant X1, mRNA 1739G>A
  source 1..1881: mol_type = genomic DNA
atgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcagtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 36 - protein, 626 aa, synthetic construct
  REGION 1..626: note = source = /note="Description of Artificial Sequence: Synthetic polypeptide"
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVQSLLEPLGLHCTFIDDFTPYHMLHGEVHCGTNVCRKPFSFKWWNMVP

SEQ ID NO: 37 - DNA, 2938 bp, Homo sapiens
  source 1..2938: mol_type = genomic DNA
agaaatggatggatgtgactgtgtgctaataaaactttatttatacaaacaggcagtaggccagatttggcccacagttcataatgtgctgatcctgacctaggcgagaagagaaaccaaatatgaaactgttgaagaacttgggactgaattatgttggaacttggtgccctgggagttaagaggagaaggtcgagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 38 - protein, 485 aa, Homo sapiens
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 39 - DNA, 2938 bp, Homo sapiens; identical to SEQ ID NO: 37
  source 1..2938: mol_type = genomic DNA

SEQ ID NO: 40 - DNA, 2938 bp, Homo sapiens; identical to SEQ ID NO: 37
  source 1..2938: mol_type = genomic DNA

SEQ ID NO: 41 - DNA, 1458 bp, Homo sapiens
  misc_feature 1..1458: note = Homo sapiens peptidyl arginine deiminase 3 (PADI3), transcript variant X2, mRNA 344C>T
  source 1..1458: mol_type = genomic DNA
atgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 42 - protein, 485 aa, synthetic construct
  REGION 1..485: note = source = /note="Description of Artificial Sequence: Synthetic polypeptide"
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 43 - DNA, 1458 bp, Homo sapiens
  misc_feature 1..1458: note = Homo sapiens peptidyl arginine deiminase 3 (PADI3), transcript variant X2, mRNA 1207G>A
  source 1..1458: mol_type = genomic DNA
atgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacgaccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 44 - protein, 485 aa, synthetic construct
  REGION 1..485: note = source = /note="Description of Artificial Sequence: Synthetic polypeptide"
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATTFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 45 - DNA, 1458 bp, Homo sapiens
  misc_feature 1..1458: note = Homo sapiens peptidyl arginine deiminase 3 (PADI3), transcript variant X2, mRNA 1276C>A
  source 1..1458: mol_type = genomic DNA
atgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggaccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 46 - protein, 485 aa, synthetic construct
  REGION 1..485: note = source = /note="Description of Artificial Sequence: Synthetic polypeptide"
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGTIINGCCCLEEKVRSLLEPLGLHCTFIDDFTPYHMLHGEVHCGTNVCRKPFSFKWWNMVP

SEQ ID NO: 47 - DNA, 1458 bp, Homo sapiens
  misc_feature 1..1458: note = Homo sapiens peptidyl arginine deiminase 3 (PADI3), transcript variant X2, mRNA 1316G>A
  source 1..1458: mol_type = genomic DNA
atgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcagtccctgctggagccgctgggcctccactgcaccttcattgatgacttcactccataccacatgctgcatggggaggtgcactgtggcaccaatgtgtgcagaaagcccttctctttcaagtggtggaacatggtgccctga

SEQ ID NO: 48 - protein, 485 aa, synthetic construct
  REGION 1..485: note = source = /note="Description of Artificial Sequence: Synthetic polypeptide"
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVQSLLEPLGLHCTFIDDFTPYHMLHGEVHCGTNVCRKPFSFKWWNMVP

SEQ ID NO: 49 - DNA, 1452 bp, Homo sapiens
  source 1..1452: mol_type = genomic DNA
cctaaggggcctcaggggcagtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccag

SEQ ID NO: 50 - protein, 421 aa, Homo sapiens
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 51 - DNA, 1266 bp, Homo sapiens
  misc_feature 1..1266: note = Homo sapiens peptidyl arginine deiminase 3 (PADI3), transcript variant X3, mRNA 154A>G
  source 1..1266: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacgtctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatga

SEQ ID NO: 52 - protein, 421 aa, synthetic construct
  REGION 1..421: note = source = /note="Description of Artificial Sequence: Synthetic polypeptide"
MSLQRIVRVSLEHPTSAVCVAGVETLVDIYGSVPEGTEMFEVYGTPGVDIYVSPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 53 - DNA, 1266 bp, Homo sapiens
  misc_feature 1..1266: note = Homo sapiens peptidyl arginine deiminase 3 (PADI3), transcript variant X3, mRNA 335T>A
  source 1..1266: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctaccacacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatga

SEQ ID NO: 54 - protein, 421 aa, synthetic construct
  REGION 1..421: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYHTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 55 - DNA, 1266 bp, Homo sapiens
  misc_feature 1..1266: note = Homo sapiens peptidyl arginine deiminase 3 (PADI3), transcript variant X3, mRNA 511G>A
  source 1..1266: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacatgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatga

SEQ ID NO: 56 - protein, 421 aa, synthetic construct
  REGION 1..421: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHMHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 57 - DNA, 1266 bp, Homo sapiens
  misc_feature 1..1266: note = Homo sapiens peptidyl arginine deiminase 3 (PADI3), transcript variant X3, mRNA 881 C>T
  source 1..1266: mol_type = genomic DNA
atgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatga

SEQ ID NO: 58 - protein, 421 aa, synthetic construct
  REGION 1..421: note = source = /note="Description of Artificial Sequence: Synthetic polypeptide"
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 59 - DNA, 1452 bp, Homo sapiens; identical to SEQ ID NO: 49
  source 1..1452: mol_type = genomic DNA

SEQ ID NO: 60 - skipped

SEQ ID NO: 61 - DNA, 2267 bp, Homo sapiens
  source 1..2267: mol_type = genomic DNA
agccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 62 - protein, 663 aa, Homo sapiens
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 63 - DNA, 1992 bp, Homo sapiens
  misc_feature 1..1992: note = Homo sapiens peptidyl arginine deiminase 4 (PADI4), transcript, mRNA 23G>A
  source 1..1992: mol_type = genomic DNA
atggcccaggggacattgatccatgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctga

SEQ ID NO: 64 - protein, 663 aa, synthetic construct
  REGION 1..663: note = source = /note="Description of Artificial Sequence: Synthetic polypeptide"
MAQGTLIHVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 65 - DNA, 1992 bp, Homo sapiens
  misc_feature 1..1992: note = Homo sapiens peptidyl arginine deiminase 4 (PADI4), transcript, mRNA 23G>T
  source 1..1992: mol_type = genomic DNA
atggcccaggggacattgatccttgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctga

SEQ ID NO: 66 - protein, 663 aa, synthetic construct
  REGION 1..663: note = source = /note="Description of Artificial Sequence: Synthetic polypeptide"
MAQGTLILVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 67 - DNA, 1801 bp, Homo sapiens
  source 1..1801: mol_type = genomic DNA
tctacctcaccggggtggaaatctccttgtgcgcagacatcacccgcaccggcaaagtgaagccaaccagagctgtgaaagatcagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 68 - DNA, 1748 bp, Homo sapiens
  source 1..1748: mol_type = genomic DNA
agctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacgagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 69 - DNA, 1806 bp, Homo sapiens
  misc_feature 1..1806: note = Homo sapiens peptidyl arginine deiminase 4 (PADI4), transcript variant X1, mRNA 23G>A
  source 1..1806: mol_type = genomic DNA
atggcccaggggacattgatccatgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctga

SEQ ID NO: 70 - protein, 601 aa, synthetic construct
  REGION 1..601: note = source = /note="Description of Artificial Sequence: Synthetic polypeptide"
MAQGTLIHVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 71 - DNA, 1806 bp, Homo sapiens
  misc_feature 1..1806: note = Homo sapiens peptidyl arginine deiminase 4 (PADI4), transcript variant X1, mRNA 23G>T
  source 1..1806: mol_type = genomic DNA
atggcccaggggacattgatccttgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctga

SEQ ID NO: 72 - protein, 601 aa, synthetic construct
  REGION 1..601: note = source = /note="Description of Artificial Sequence: Synthetic polypeptide"
MAQGTLILVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 73 - DNA, 1692 bp, Homo sapiens
  misc_feature 1..1692: note = Homo sapiens peptidyl arginine deiminase 4 (PADI4), transcript variant X2, mRNA 23G>A
  source 1..1692: mol_type = genomic DNA
atggcccaggggacattgatccatgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctga

SEQ ID NO: 74 - protein, 563 aa, synthetic construct
  REGION 1..563: note = source = /note="Description of Artificial Sequence: Synthetic polypeptide"
MAQGTLIHVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 75 - DNA, 1692 bp, Homo sapiens
  misc_feature 1..1692: note = Homo sapiens peptidyl arginine deiminase 4 (PADI4), transcript variant X2, mRNA 23G>T
  source 1..1692: mol_type = genomic DNA
atggcccaggggacattgatccttgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctga

SEQ ID NO: 76 - protein, 563 aa, synthetic construct
  REGION 1..563: note = source = /note="Description of Artificial Sequence: Synthetic polypeptide"
MAQGTLILVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 77 - DNA, 1194 bp, Homo sapiens
  misc_feature 1..1194: note = Homo sapiens peptidyl arginine deiminase 4 (PADI4), transcript variant X4, mRNA 23G>A
  source 1..1194: mol_type = genomic DNA
atggcccaggggacattgatccatgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgattctatcaataggcccattttatagaagaagaaactga

SEQ ID NO: 78 - protein, 397 aa, synthetic construct
  REGION 1..397: note = source = /note="Description of Artificial Sequence: Synthetic polypeptide"
MAQGTLIHVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMILSIGPFYRRRN

SEQ ID NO: 79 - DNA, 1194 bp, Homo sapiens
  misc_feature 1..1194: note = Homo sapiens peptidyl arginine deiminase 4 (PADI4), transcript variant X4, mRNA 23G>T
  source 1..1194: mol_type = genomic DNA
atggcccaggggacattgatccttgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgattctatcaataggcccattttatagaagaagaaactga

SEQ ID NO: 80 - protein, 397 aa, synthetic construct
  REGION 1..397: note = source = /note="Description of Artificial Sequence: Synthetic polypeptide"
MAQGTLILVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMILSIGPFYRRRN

SEQ ID NO: 81 - DNA, 1179 bp, Homo sapiens
  misc_feature 1..1179: note = Homo sapiens peptidyl arginine deiminase 4 (PADI4), transcript variant X6, mRNA 23G>A
  source 1..1179: mol_type = genomic DNA
atggcccaggggacattgatccatgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatggaagaccccaggcctgcgctctga

SEQ ID NO: 82 - protein, 392 aa, synthetic construct
  REGION 1..392: note = source = /note="Description of Artificial Sequence: Synthetic polypeptide"
MAQGTLIHVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMEDPRPAL

SEQ ID NO: 83 - DNA, 1179 bp, Homo sapiens
  misc_feature 1..1179: note = Homo sapiens peptidyl arginine deiminase 4 (PADI4), transcript variant X6, mRNA 23G>T
  source 1..1179: mol_type = genomic DNA
atggcccaggggacattgatccttgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatggaagaccccaggcctgcgctctga

SEQ ID NO: 84 - protein, 392 aa, synthetic construct
  REGION 1..392: note = source = /note="Description of Artificial Sequence: Synthetic polypeptide"
MAQGTLILVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMEDPRPAL

SEQ ID NO: 85 - DNA, 1161 bp, Homo sapiens
  misc_feature 1..1161: note = Homo sapiens peptidyl arginine deiminase 4 (PADI4), transcript variant X7, mRNA 23G>A
  source 1..1161: mol_type = genomic DNA
atggcccaggggacattgatccatgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgcaatga

SEQ ID NO: 86 - protein, 386 aa, synthetic construct
  REGION 1..386: note = source = /note="Description of Artificial Sequence: Synthetic polypeptide"
MAQGTLIHVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMQ

SEQ ID NO: 87 - DNA, 1161 bp, Homo sapiens
  misc_feature 1..1161: note = Homo sapiens peptidyl arginine deiminase 4 (PADI4), transcript variant X7, mRNA 23G>T
  source 1..1161: mol_type = genomic DNA
atggcccaggggacattgatccttgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgcaatga

SEQ ID NO: 88 - protein, 386 aa, synthetic construct
  REGION 1..386: note = source = /note="Description of Artificial Sequence: Synthetic polypeptide"
MAQGTLILVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMQ

SEQ ID NO: 89 - DNA, 1992 bp, Homo sapiens; identical to SEQ ID NO: 63
  misc_feature 1..1992: note = Homo sapiens peptidyl arginine deiminase 4 (PADI4), transcript variant X5, mRNA 23G>A
  source 1..1992: mol_type = genomic DNA

SEQ ID NO: 90 - protein, 397 aa, synthetic construct; identical to SEQ ID NO: 78
  REGION 1..397: note = source = /note="Description of Artificial Sequence: Synthetic polypeptide"

SEQ ID NO: 91 - DNA, 1992 bp, Homo sapiens; identical to SEQ ID NO: 65
  misc_feature 1..1992: note = Homo sapiens peptidyl arginine deiminase 4 (PADI4), transcript variant X5, mRNA 23G>T
  source 1..1992: mol_type = genomic DNA

SEQ ID NO: 92 - protein, 397 aa, synthetic construct; identical to SEQ ID NO: 80
  REGION 1..397: note = source = /note="Description of Artificial Sequence: Synthetic polypeptide"

(19) United States
(12) Patent Application Publication
(10) Pub. No.: US 2023/0118822 A1
(43) Pub. Date: Apr. 20, 2023

(54) COMPOSITIONS AND METHODS FOR DIAGNOSING AND ASSESSING RHEUMATOID ARTHRITIS USING PROTEIN-ARGININE DEIMINASE 1 (PAD1) AUTOANTIGENS

(71) Applicant: INOVA DIAGNOSTICS, INC., SAN DIEGO, CA (US)

(72) Inventors: Michael MAHLER, San Diego, CA (US); Laura MARTINEZ-PRAT, San Diego, CA (US); Chelsea BENTOW, San Diego, CA (US)

(73) Assignee: INOVA DIAGNOSTICS, INC., SAN DIEGO, CA (US)

(21) Appl. No.: 17/961,412

(22) Filed: Oct. 6, 2022

Related U.S. Application Data
(63) Continuation of application No. PCT/US2021/032471, filed on May 14, 2021.
(60) Provisional application No. 63/025,854, filed on May 15, 2020.

(51) Int. Cl. G01N 33/564

(52) U.S. Cl. CPC G01N 33/564; G01N 2333/978; G01N 2800/102; G01N 2800/52

(57) ABSTRACT

The present disclosure relates to the use of PAD proteins, such as PAD1, or PAD1 and PAD4, or antigenic fragments thereof as clinical biomarker for diagnostic and prognostic information in rheumatoid arthritis (RA) patients. The disclosure further provides methods and compositions for the detection of autoantibodies against PAD proteins, such as anti-PAD1, or anti-PAD1 and anti-PAD4, in a biological sample.

This application is a continuation of PCT/US2021/032471, filed May 14, 2021, which PCT Application claims the benefit of U.S. Provisional Application No. 63/025,854, filed May 15, 2020, both of which are hereby incorporated herein by reference in their entireties.

The instant application contains a Sequence Listing which has been submitted electronically in ASCII format and is hereby incorporated by reference in its entirety. Said ASCII copy, created on May 14, 2021, is named 13510-039-228_SL.txt and is 336,532 bytes in size.

### FIELD

The present disclosure generally relates to the fields of molecular and cellular biology and immunology, and more specifically to methods for detecting autoantibodies against one or more PAD proteins, such as PAD1, or combinations of PAD1 and other PAD proteins, or antigenic fragments thereof in the serum of rheumatoid arthritis (RA) patients. Also provided herein are methods of diagnosing a patient having or suspected of having RA.

### BACKGROUND

Rheumatoid Arthritis (RA) is a chronic autoimmune disease characterized by inflammation, pain and subsequent damage to synovial-lined joints. Unlike other arthritis conditions, RA is a systemic disease that can affect other organ systems including but not limited to the cardiovascular system, the respiratory system and musculature. While the exact pathogenesis of the disease is unknown, RA is characterized by the production of antibodies to self-proteins (autoantibodies) by the immune system. The most common autoantibodies implicated in RA include rheumatoid factor (RF) and anti-citrullinated protein antibodies (ACPAs), which are part of the classification criteria for this disease. ACPAs are a hallmark amongst serologic factors detected in RA patients, and as such, serve as valuable diagnostic and prognostic markers. (See, e.g., Aletaha D. et al., Ann. Rheum. Dis. 2010, 69, 1580-1588; Taylor et al., Autoimmune Dis; 2011:815038 (2011)). However, clinical heterogeneity of RA precludes the use of ACPAs and RF alone as reliable biomarkers. Patients with erosive disease require more aggressive treatment in the early phase of the disease to prevent joint damage. More precise biomarkers that specifically identify sufferers of RA and disease progression are needed.

Protein-arginine deiminases (PADs) are calcium-dependent enzymes that play a central role in generating autoantigens in RA through the conversion of arginine residues to citrulline, a process known as citrullination. Beyond ACPA and RF, autoantibodies which target the PAD enzymes, have also been described in RA, (see, e.g., Takizawa et al., Scand. J. Rheumatol. 3: 212-215 (2005); Roth et al., Clin. Exp. Rheumatol. 1: 12-18 (2006); Halvorsen et al., Ann. Rheumatol. Dis. 67:414-417 (2008); Zhao et al., J. Rheumatol., 35:969-974 (2008); Darrah et al., Sci. Trans. Med., 5(186):186ra65 (2013); Darrah et al., Front. Immunol., 9:2696 (2018)). As such, PADs appear to play a central role in RA pathogenesis.

A total of five members of the PAD family have been reported in humans: PAD1, 2, 3, 4, and 6. Among the five PAD proteins, PAD2 and PAD4 are known to play a central role in the pathogenesis of rheumatoid arthritis (RA) and, together with PAD3, they have also been identified as antigenic targets (see, e.g., Curran, A. M., et al., Nature Reviews Rheumatology, 2020; Darrah, E., et al., Ann Rheum Dis, 2012. 71(1): p. 92-8). In particular, the detection of antibodies against PAD4 (anti-PAD4) is associated with markers of disease severity and patients with worse baseline radiographic joint damage (see, e.g., Darrah E, et al. J Rheumatol., 46:329-330 (2019)), whereas detection of antibodies against PAD2 (anti-PAD2) are associated with fewer swollen joints and less interstitial lung disease (ILD) in RA (see, e.g., Darrah et al., Front. Immunol., 9:2696 (2018)).

Although the detection of anti-PAD4 is strongly associated with RA, having a specificity of roughly 96%, anti-PAD4 antibodies are usually found in a subgroup of RA patients with a prevalence of 20-45% (see, e.g., Ren J., et al., Clinical rheumatology, 36:2431-2438 (2017)). Detection of anti-PAD2 is generally not associated with anti-PAD4, and is found in approximately 18.5% of RA patients. Thus, there exists an unmet need for additional biomarkers for the diagnosis of RA and assessment of disease progression. The present disclosure satisfies this need and provides related advantages as well.

### SUMMARY

In one aspect, provided herein is a method of diagnosing rheumatoid arthritis (RA), comprising: (a) contacting a biological sample from a subject suspected of having RA with at least one peptidyl arginine deiminase (PAD) protein or an antigenic fragment thereof, and (b) detecting the presence of an autoantibody reactive with the at least one PAD protein or an antigenic fragment thereof, wherein the presence of said autoantibody is indicative of RA, wherein the at least one PAD protein comprises PAD1, or PAD1 and PAD4.

In certain embodiments, the at least one PAD protein is PAD1 or an antigenic fragment thereof. In other embodiments, the at least one PAD protein is PAD1 and PAD4 or an antigenic fragment thereof.

In some embodiments, the at least one PAD protein further comprises one or more PAD protein selected from the group consisting of PAD2, PAD3, and PAD6 or an antigenic fragment thereof. In specific embodiments, the at least one PAD protein is PAD1, PAD4, and PAD2 or an antigenic fragment thereof. In other embodiments, the at least one PAD protein is PAD1, PAD4, and PAD3 or an antigenic fragment thereof. In further embodiments, the at least one PAD protein is PAD1, PAD4, PAD2, and PAD3 or an antigenic fragment thereof. In still further embodiments, the at least one PAD protein is PAD1, PAD4, PAD2, PAD3, and PAD6 or an antigenic fragment thereof.

Also provided herein is a method of monitoring the progression of rheumatoid arthritis (RA), comprising: (a) contacting a biological sample from a subject having or suspected of having RA with at least one peptidyl arginine deiminase (PAD) protein or an antigenic fragment thereof, and (b) detecting the presence of an autoantibody reactive with the at least one PAD protein or an antigenic fragment thereof, wherein the presence of said autoantibody is indicative of disease progression, wherein the at least one PAD protein comprises PAD1, or PAD1 and PAD4.

In some embodiments, the at least one PAD protein is PAD1 or an antigenic fragment thereof. In other embodiments, the at least one PAD protein is PAD1 and PAD4 or an antigenic fragment thereof. In certain embodiments, the at least one PAD protein further comprises PAD3 or an antigenic fragment thereof. In some embodiments, the presence of said autoantibody is indicative of RA stage.

The present disclosure also provides a method of monitoring the progression of rheumatoid arthritis (RA), comprising: (a) contacting a biological sample from a subject having RA with at least one peptidyl arginine deiminase (PAD) protein or an antigenic fragment thereof, and (b) detecting the absence of an autoantibody bound to the at least one PAD protein or an antigenic fragment thereof, wherein the absence of said autoantibody is indicative of disease progression, wherein the at least one PAD protein comprises PAD1, or PAD1 and PAD4.

In some embodiments, the at least one PAD protein is PAD1 or an antigenic fragment thereof. In other embodiments, the at least one PAD protein is PAD1 and PAD4 or an antigenic fragment thereof. In certain embodiments, the at least one PAD protein further comprises PAD3 or an antigenic fragment thereof. In some embodiments, the presence of said autoantibody is indicative of RA stage.

In some embodiments of the present disclosure the biological sample comprises whole blood, serum, plasma synovial fluid or sputum. In specific embodiments the biological sample comprises serum or plasma.

In some aspects of the present disclosure the antigenic fragment comprises from 6-120, 12-100, 18-80, 24-60, 30-50 or 35-45 amino acid residues.

In some embodiments, the PAD protein or antigenic fragment thereof is obtained by a method comprising isolation from a natural source, chemical synthesis or recombinant expression. In specific embodiments, the PAD protein or antigenic fragment thereof is obtained by chemical synthesis.

As provided herein, in some embodiments the detection comprises an immunoassay. In specific embodiments, the immunoassay is selected from the group consisting of a fluorescent immunosorbent assay (FIA), a chemiluminescent immunoassay (CIA), a radioimmunoassay (RIA), multiplex immunoassay, a protein/peptide array immunoassay, a solid phase radioimmunoassay (SPRIA), an indirect immunofluorescence assay (IIF), an enzyme linked immunosorbent assay (ELISA), a particle based multianalyte test (PMAT), and a Dot Blot assay.

In some embodiments, detection comprises contacting said autoantibody bound to the PAD protein or antigenic fragment thereof with a detection probe. In certain embodiments, the detection probe binds to said autoantibody. In some embodiments, the detection probe comprises an antibody or functional fragment thereof. In other embodiments, the detection probe comprises a reporter tag.

In certain embodiments, the reporter tag is a label. In specific embodiments, the label is selected from the group consisting of a fluorophore, enzyme, chemiluminescent moiety, radioactive moiety, organic dye and small molecule.

In some embodiments, the label is a fluorescent label. In specific embodiments, the fluorescent label is phycoerytherin (PE).

In some embodiments, the reporter tag comprises a ligand or a particle. In certain embodiments, the ligand is biotin. In some embodiments, the particle comprises a nanoparticle.

Also provided herein is a detection kit that includes at least one peptidyl arginine deiminase (PAD) protein, or an antigenic fragment thereof, that can capture an autoantibody specific to the PAD protein; a detection probe that recognizes said autoantibody, and a solid support, wherein the at least one PAD protein comprises PAD1, or PAD1 and PAD4.

In certain embodiments, the at least one PAD protein is PAD1 or an antigenic fragment thereof. In some embodiments, the at least one PAD protein is PAD1 and PAD4 or an antigenic fragment thereof.

In some embodiments, the at least one PAD protein further comprises one or more PAD protein selected from the group consisting of PAD2, PAD3, and PAD6 or an antigenic fragment thereof. In specific embodiments, the at least one PAD protein is PAD1, PAD4, and PAD2 or an antigenic fragment thereof. In other embodiments, the at least one PAD protein is PAD1, PAD4, and PAD3 or an antigenic fragment thereof. In further embodiments, the at least one PAD protein is PAD1, PAD4, PAD2, and PAD3 or an antigenic fragment thereof. In yet other embodiments, the at least one PAD protein is PAD1, PAD4, PAD2, PAD3, and PAD6 or an antigenic fragment thereof.

In some embodiments, the kit further comprises a label. In some embodiments, the label is selected from the group consisting of a fluorophore, enzyme, chemiluminescent moiety, radioactive moiety, organic dye and small molecule.

In some embodiments, the kit further comprises a positive control.

In some embodiments the kit further comprises one or more ancillary reagents. In specific embodiments, the one or more ancillary reagents is selected from the group consisting of an incubation buffer, a wash buffer, a detection buffer and a detection instrument.

In some embodiments, the antigenic fragment comprises from 6-120, 12-100, 18-80, 24-60, 30-50 or 35-45 amino acid residues.

In some embodiments, the detection probe comprises an antibody or functional fragment thereof. In other embodiments, the detection probe comprises a reporter tag. In specific embodiments, the reporter tag is a label. In some embodiments, the label is selected from the group consisting of a fluorophore, enzyme, chemiluminescent moiety, radioactive moiety, organic dye and small molecule.

In some embodiments the label is a fluorescent label. In specific embodiments, the fluorescent label is phycoerytherin (PE).

In some embodiments the reporter tag comprises a ligand or particle. In specific embodiments, the ligand is biotin. In some embodiments, the particle comprises a nanoparticle.

In some embodiments, the solid support is selected from the group consisting of a bead, sphere, particle, membrane, chip, slide, plate, well and test tube. In specific embodiments, the bead, sphere or particle has a diameter of about 0.1 to about 100 micrometer. In some embodiments, the membrane is selected from the group consisting of nitrocellulose, nylon, polyvinylidene fluoride (PVDF) and polyvinylidene difluoride.

In some embodiments, the PAD protein or antigenic fragment thereof is conjugated to said solid support.

### BRIEF DESCRIPTION OF THE DRAWINGS

FIG. 1 shows a receiver operating characteristic (ROC) analysis of anti-PAD1 IgG (closed circle), anti-PAD2 IgG (closed dark grey square), anti-PAD3 IgG (triangle), anti-PAD4 IgG (open circle), and anti-PAD6 IgG (light grey square) illustrating the discrimination between RA and non-RA patients from Cohort I. Solid line shows no discrimination. Area Under the Curve (AUC) for each marker is shown in the legend. Abbreviations: TPF: true positive fraction; FPF: false positive fraction.

FIG. 2 shows a receiver operating characteristic (ROC) analysis of anti-PAD1 (open circle), anti-PAD2 (closed circle), anti-PAD3 (open triangle), anti-PAD4 (grey square), and anti-PAD6 (black grey square) illustrating the discrimination between RA and non-RA patients from Cohort II. Solid line shows no discrimination. Area Under the Curve (AUC) for each marker is shown in the legend. Abbreviations: TPF: true positive fraction; FPF: false positive fraction.

FIG. 3 shows high specificity for anti-PAD1 antibodies in discriminating RA patients from non-RA patients. Non-RA controls included samples from Hashimoto's disease (HD), idiopathic inflammatory myopathies (IIM), Sjögren's syndrome (SjS), ankylosing spondylitis (AS), healthy individuals (HI), juvenile idiopathic arthritis (JIA), psoriatic arthritis (PsA), systemic lupus erythematosus (SLE), chronic obstructive pulmonary disease (COPD), infectious diseases (ID), osteoarthritis (OA), and small vessel vasculitis (SVV).

FIG. 4 shows a two dimensional principal component analysis (PCA) plot of the anti-PAD levels in RA patients (n=33) and controls (n=36). Anti-PAD1, anti-PAD3 and anti-PAD4 have the main contribution to PC1, which explains 51.7% of the variance, and anti-PAD2 and anti-PAD6 to PC2, that represents 20.8% of it. Abbreviations: PC: principal component.

FIG. 5 shows the correlation between anti-PAD1 and anti-PAD4, with some samples from Cohort II that react with PAD1 or PAD4 with high levels.

FIG. 6. Shows a receiver operating characteristic (ROC) analysis of anti-PAD1, anti-PAD4, anti-PAD1/anti-PAD4, and anti-PAD1/anti-PAD2/anti-PAD6 illustrating the discrimination between RA and non-RA patients. Solid line shows no discrimination. Area Under the Curve (AUC) for each marker is shown in the legend. Abbreviations: TPF: true positive fraction; FPF: false positive fraction.

FIG. 7A, FIG. 7B and FIG. 7C show a receiver operating characteristics (ROC) analysis (FIG. 7A) and likelihood ratio plots (FIG. 7B) for anti-PAD1 IgA and anti-PAD4 IgA. A total of 51 RA patients and 15 controls were tested to assess the ability to discriminate RA from controls for anti-PAD1 IgA and anti-PAD4 IgA. FIG. 7A shows the ROC curve for the two antigens and indicates equal or superior performance for anti-PAD1 IgA vs. anti-PAD4 IgA. FIG. 7B shows the likelihood and odds ratios (OR) for both anti-PAD1 IgA (left) and anti-PAD4 IgA (right, FIG. 7C). Abbreviations: TPF: true positive fraction; FPF: false positive fraction

FIG. 8A and FIG. 8B show correlation between anti-PAD autoantibodies. FIG. 8A shows the correlation between anti-PAD1 IgA and anti-PAD1 IgG. Although a significant correlation was observed, individual patients had varying levels of anti-PAD1 IgA and anti-PAD1 IgG. FIG. 8B shows the correlation between anti-PAD1 IgA and anti-PAD4 IgA.

FIG. 9A and FIG. 9B show sodium dodecyl sulphate-polyacrylamide gel electrophoresis (SDS-PAGE) (FIG. 9A) and anti-modified citrulline (AMC) immunoblot (FIG. 9B) analysis of the different PAD antigens including the PAD1 proteins generated in-house in the absences or presence of calcium, as well as other commercial PADs and different experimental controls. The molecular weights associated to each band in the protein ladder are shown on the left of the gel and the blot.

### DETAILED DESCRIPTION

The present disclosure is based, in part, on the discovery that PAD1 is a novel autoantigen in RA, and that detection of autoantibodies against PAD1 (“anti-PAD1”) serves as a diagnostic biomarker for RA. Aspects of the present disclosure are also based, in part, on the discovery that detection of anti-PAD1 can be combined with detection of one or more anti-PAD autoantibodies, such as anti-PAD4, to increase the sensitivity for discriminating between RA and non-RA patients. Thus, the present disclosure benefits RA patients by providing new biomarkers that can indicate the presence of RA.

Unless particularly defined otherwise, all terms including technical and scientific terms used in this application have the same meaning as commonly understood by one of ordinary skill in the art to which this invention belongs. In general, the nomenclatures used in this specification and the experimental methods described below are widely known and generally used in the related art.

For purposes of interpreting this specification, the following description of terms will apply and whenever appropriate, terms used in the singular will also include the plural and vice versa. It must also be noted that, as used in this specification and the appended claims, where a range of numeric values is provided, it is understood that the ranges are inclusive of the numbers defining the range. It is also understood that each intervening integer within the recited range as well as fractions thereof, including for example, every tenth of a unit of a selected intervening integer or a lower limit of the recited range is intended to be included within the disclosure, unless the context clearly dictates otherwise.

As used herein, the terms “comprises,” “comprising,” “includes,” “including,” “has,” “having,” “contains,” “containing,” and any variations thereof, are intended to cover a non-exclusive inclusion, such that a process, method, product-by-process, or composition of matter that includes has or contains an element or list of elements, does not include only those elements but can include other elements not expressly listed or inherent to such process, method, product-by-process, or composition of matter.

As used herein, the term “autoantibody” is intended to mean an immunoglobulin molecule that binds an autoimmune antigen or epitope thereof, such as a self-protein, carbohydrate, nucleic acid or other molecule present in the autoantibody producing animal. The antibodies can be from any animal origin including, for example, mammals such as human, murine, rabbit, goat, guinea pig, camel, horse and the like. Generally, an animal immune system is able to recognize and ignore the body's own healthy proteins, cells, and tissues. Sometimes, however, the immune system ceases to recognize one or more of the body's normal constituents as “self,” leading to production of autoantibodies that can result in certain pathologies such as inflammation and tissue damage.

As used herein, the term “antigenic fragment” is intended to mean a portion of an antigen. The term includes 6-120, 12-100, 18-80, 24-60, 30-50 or 35-45 amino acid residues.

As used herein, the term “peptidyl arginine deiminase” or “PAD,” also known as PAD1, refers to a family of enzymes that catalyze the post-translational modifications of protein arginine residues by deimination or demethylimination to produce citrulline (see, e.g., Wang and Wang, Biochim. Biophys. Acta., 1829:1126-35 (2013)). Five isotypes of PADs have been identified in humans and include PAD1, PAD2, PAD3, PAD4 and PAD6. All of such PAD polypeptides, PAD1, PAD2, PAD3, PAD4 and PAD6 are included within the meaning of the term “PAD” as it is used herein.

As used herein, there term “reactive” when used in reference to an autoantibody and a PAD protein or an antigenic fragment thereof is intended to me that the autoantibody specifically recognizes the PAD protein or antigenic fragment thereof. Generally this will involve binding or otherwise interacting with the PAD protein or an antigenic fragment thereof to form an antigen-antibody complex. As such, autoantibodies that are reactive with the PAD protein or an antigenic fragment thereof are understood to be unique for the PAD protein or an antigenic fragment and an autoantibody for a particular PAD protein will only be reactive with that particular PAD protein.

As used herein, reference to RA stage is intended to refer to the four main stages of RA, categorized by clinical and radiologic criteria. Stage I (early RA) generally involves no destructive changes observed upon radiographic examination, and may involve initial inflammation in the joint capsule and swelling of synovial tissue. Stage II (moderate progression) generally involves radiographic evidence of periarticular osteoporosis, with or without slight subchondral bone destruction; slight cartilage destruction is possible; joint mobility is possibly limited, but no joint deformities are observed; adjacent muscle atrophy is present; extra-articular soft tissue lesions (e.g., nodules and tenosynovitis) are possible. Stage III (severe progression) generally involves radiographic evidence of cartilage and bone destruction in addition to periarticular osteoporosis; joint deformity (e.g., subluxation, ulnar deviation, or hyperextension) without fibrous or bony ankylosis; muscle atrophy is extensive; extra-articular soft tissue lesions (e.g., nodules, tenosynovitis) are possible. Stage IV (terminal progression) generally involves the presence of fibrous or bony ankylosis, along with criteria of stage III.

As used herein, the term “solid support” is intended to mean any material that can serve as a solid or semi-solid foundation for the deposition of one or more of autoimmune antigens or fragments thereof for use in detecting autoantibodies. Representative examples of solid supports include, for example, beads, particles including microparticles and nanoparticles, wells of micro- or multi-well plates, gels, colloids, sheet, chip, electrodes, test tubes, and other configurations known to those of ordinary skill in the art. Representative particles include, for example, beads, spheres or other solid support carrier.

As used herein, the term “ancillary agent” is intended to mean a reagent or component applicable in a detection method. Ancillary reagents can include, e.g., an immobilization buffer, an immobilization reagent, a dilution buffer, a secondary antibody, a reporter reagent, a detection reagent, a blocking buffer, a washing buffer, a detection buffer, a stop solution, a system rinse buffer, a system cleaning solution, or any combination thereof.

The protein-arginine deiminase (PAD) enzymes were described for the first time in 1977. A total of five members of the PAD family have been reported in humans: PAD1, 2, 3, 4, and 6, with significant protein sequence homology between them. Among the five PAD proteins, PAD2 and PAD4 are known to play a central role in the pathogenesis of rheumatoid arthritis (RA) and, together with PAD3, they have also been identified as antigenic targets. However, little is known about PAD1 or PAD6.

As used herein, the term “peptidyl arginine deiminase 1” or “PAD1,” also known as PAD11, and PDI1, refers to a member of the PAD family of enzymes.

As used herein, the term “peptidyl arginine deiminase 2” or “PAD2,” also known as PAD12, PAD-H19 and PDI2, refers to a member of the PAD family of enzymes. PAD2 is abundantly expressed in secretory glands, brain, uterus, spleen, pancreas and skeletal muscle. Known substrates of PAD2 include myelin basic protein, vimentin and macrophages. See Vossenaar et al., Annals of the Rheumatic Diseases, 63:373-81 (2004); Watanbe et al., Biochim Biophys Acta., 966:375-383 (1988); Watanabe et al., J. Biol Chem., 264:15255-15260 (1989); Nagata et al., Experientia, 46:72-74 (1990); Urano et al., Am J Dermatopathol., 12(3):249-55 (1990), Vossenaar et al., Arthritis and Rheum., 48:2489-2500 (2003). Approximately 726 coding single nucleotide polymorphisms (SNP) have been identified for PAD2 and at least 184 known orthologs (see, for example, NCBI Gene ID:11240). The term “PAD2” includes all of such PAD2 variants and PAD2 orthologs. An exemplary human PAD2 (hPAD2) nucleotide sequence can be found in GenBank under GenBank Accession number NM_007365 (SEQ ID NO:1) and encodes an exemplary human PAD2 having the amino acid sequence found under found under GenBank Accession number NP_031391 (SEQ ID NO:2). The GenBank Accession numbers and GenBank GI numbers of this PAD2 and other exemplary PAD2 enzymes can be found below in Table 1. All of such PAD2 polypeptides and variants thereof are included within the meaning of the term “PAD2” as it is used herein.

In some embodiments, a PAD2, or antigenic fragment thereof, includes the amino acid in SEQ ID NO:2, of a mature human PAD2 (hPAD2; amino acids 25-665 of NCBI Accession Number NP_031391; GI: 122939159), or naturally occurring variants thereof:

SEQ ID NO: 2
MLRERTVRLQYGSRVEAVYVLGTYLWTDVYSAAPAGAQTFSLKHSEHVWV
EVVRDGEAEEVATNGKQRWLLSPSTTLRVTMSQASTEASSDKVTVNYYDE
EGSIPIDQAGLFLTAIEISLDVDADRDGVVEKNNPKKASWTWGPEGQGAI
LLVNCDRETPWLPKEDCRDEKVYSKEDLKDMSQMILRTKGPDRLPAGYEI
VLYISMSDSDKVGVFYVENPFFGQRYIHILGRRKLYHVVKYTGGSAELLF
FVEGLCFPDEGFSGLVSIHVSLLEYMAQDIPLTPIFTDTVIFRIAPWIMT
PNILPPVSVFVCCMKDNYLFLKEVKNLVEKTNCELKVCFQYLNRGDRWIQ
DEIEFGYIEAPHKGFPVVLDSPRDGNLKDFPVKELLGPDFGYVTREPLFE
SVTSLDSFGNLEVSPPVTVNGKTYPLGRILIGSSFPLSGGRRMTKVVRDF
LKAQQVQAPVELYSDWLTVGHVDEFMSFVPIPGTKKFLLLMASTSACYKL
FREKQKDGHGEAIMFKGLGGMSSKRITINKILSNESLVQENLYFQRCLDW
NRDILKKELGLTEQDIIDLPALFKMDEDHRARAFFPNMVNMIVLDKDLGI
PKPFGPQVEEECCLEMHVRGLLEPLGLECTFIDDISAYHKFLGEVHCGTN
VRRKPFTFKWWHMVP.

As used herein, the term “peptidyl arginine deiminase 3” or “PAD3,” also known as PAD13, PDI3 and UHS1, refers to a member of the PAD family of enzymes. PAD3 is detected in the epidermis where it plays a role in terminal differentiation and in hair follicles where it modulates structural proteins including filaggrin and trichoyalin. See Chavanas et al., J Dermatol Sci., 44:63-72 (2006); Kanno et al., J. Invest Dermatol. 115(5):813-23 (2000); Nachat et al., J Investig Dermatol., 125:34-41 (2005). Approximately 738 coding SNPs have been identified for PAD3 and at least 109 known orthologs (see, for example, NCBI Gene ID: 51702). The term “PAD3” includes all of such PAD3 variants and PAD3 orthologs. An exemplary human PAD3 (hPAD3) nucleotide sequence can be found in GenBank under GenBank Accession number NM_016233 (SEQ ID NO:5) and encodes an exemplary human PAD3 having the amino acid sequence found under found under GenBank Accession number NP_057317 (SEQ ID NO:6). The GenBank Accession numbers and GenBank GI numbers of this PAD3 and other exemplary PAD3_enzymes can be found below in Table 2. All of such PAD3 polypeptides and variants thereof are included within the meaning of the term “PAD3” as it is used herein.

In some embodiments, a PAD3, or antigenic fragment thereof, includes the amino acid in SEQ ID NO:6 of a mature human PAD3 (hPAD3; amino acids 25-664 of NCBI Accession Number NP_057317; GI: 122939161), or naturally occurring variants thereof:

SEQ ID NO: 6
MSLQRIVRVSLEHPTSAVCVAGVETLVDIYGSVPEGTEMFEVYGTPGVDI
YISPNMERGRERADTRRWRFDATLEIIVVMNSPSNDLNDSHVQISYHSSH
EPLPLAYAVLYLTCVDISLDCDLNCEGRQDRNFVDKRQWVWGPSGYGGIL
LVNCDRDDPSCDVQDNCDQHVHCLQDLEDMSVMVLRTQGPAALFDDHKLV
LHTSSYDAKRAQVFHICGPEDVCEAYRHVLGQDKVSYEVPRLHGDEERFF
VEGLSFPDAGFTGLISFHVTLLDDSNEDFSASPIFTDTVVFRVAPWIMTP
STLPPLEVYVCRVRNNTCFVDAVAELARKAGCKLTICPQAENRNDRWIQD
EMELGYVQAPHKTLPVVFDSPRNGELQDFPYKRILGPDFGYVTREPRDRS
VSGLDSFGNLEVSPPVVANGKEYPLGRILIGGNLPGSSGRRVTQVVRDFL
HAQKVQPPVELFVDWLAVGHVDEFLSFVPAPDGKGFRMLLASPGACFKLF
QEKQKCGHGRALLFQGVVDDEQVKTISINQVLSNKDLINYNKFVQSCIDW
NREVLKRELGLAECDIIDIPQLFKTERKKATAFFPDLVNMLVLGKHLGIP
KPFGPIINGCCCLEEKVRSLLEPLGLHCTFIDDFTPYHMLHGEVHCGTNV
CRKPFSFKWWNMVP

As used herein, the term “peptidyl arginine deiminase 4” or “PAD4,” also known as PAD, PAD14, PDI4, PADV, PDI5 and PAD15, refers to a member of the PAD family of enzymes. PAD4 can be detected in white blood cells including granulocytes and monocytes under normal physiological conditions (Vossenaar et al., Annals of the Rheumatic Diseases, 63:373-81 (2004); Asaga et al., J. Leukocyte Biology 70:46-51 (2001)) and is generally localized in the nucleus (Nakashima et al., JBC 277:49562-68 (2002)). Approximately 737 coding SNPs have been identified for PAD4 and at least 108 known orthologs (see, for example, NCBI Gene ID:23569). The term “PAD4” includes all of such PAD4 variants and PAD4 orthologs. An exemplary human PAD4 (hPAD4) nucleotide sequence can be found in GenBank under GenBank Accession number NM_012387.2 (SEQ ID NO:61) and encodes an exemplary human PAD4 having the amino acid sequence found under found under GenBank Accession number NP_036519.2 (SEQ ID NO:62). The GenBank Accession numbers and GenBank GI numbers of this PAD4 and other exemplary PAD4 enzymes can be found below in Table 3. All of such PAD4 polypeptides and variants thereof are included within the meaning of the term “PAD4” as it is used herein.

In some embodiments, a PAD4, or antigenic fragment thereof, includes the amino acid in SEQ ID NO:62, of a mature human PAD4 (hPAD4; amino acids 25-663 of NCBI Accession Number NP_036519; GI: 216548487), or naturally occurring variants thereof:

SEQ ID NO: 62
MAQGTLIRVTPEQPTHAVCVLGTLTQLDICSSAPEDCTSFSINASPGVVV
DIAHGPPAKKKSTGSSTWPLDPGVEVTLTMKVASGSTGDQKVQISYYGPK
TPPVKALLYLTGVEISLCADITRTGKVKPTRAVKDQRTWTWGPCGQGAIL
LVNCDRDNLESSAMDCEDDEVLDSEDLQDMSLMTLSTKTPKDFFTNHTLV
LHVARSEMDKVRVFQATRGKLSSKCSVVLGPKWPSHYLMVPGGKHNMDFY
VEALAFPDTDFPGLITLTISLLDTSNLELPEAVVFQDSVVFRVAPWIMTP
NTQPPQEVYACSIFENEDFLKSVTTLAMKAKCKLTICPEEENMDDQWMQD
EMEIGYIQAPHKTLPVVFDSPRNRGLKEFPIKRVMGPDFGYVTRGPQTGG
ISGLDSFGNLEVSPPVTVRGKEYPLGRILFGDSCYPSNDSRQMHQALQDF
LSAQQVQAPVKLYSDWLSVGHVDEFLSFVPAPDRKGFRLLLASPRSCYKL
FQEQQNEGHGEALLFEGIKKKKQQKIKNILSNKTLREHNSFVERCIDWNR
ELLKRELGLAESDIIDIPQLFKLKEFSKAEAFFPNMVNMLVLGKHLGIPK
PFGPVINGRCCLEEKVCSLLEPLGLQCTFINDFFTYHIRHGEVHCGTNVR
RKPFSFKWWNMVP

Tables 1, 2 and 3 contain two sequence identifiers, the GI number and the GenBank accession number. The GI number and GenBank accession number run in parallel as unique identifiers to access the referenced sequence in publicly available databases. Table 1 includes GI numbers and GenBank Accession numbers for PAD2, Table 2 includes GI numbers and GenBank Accession numbers for PAD3 and Table 3 includes GI numbers and GenBank accession numbers for PAD4.

The sequence identifiers in Tables 1, 2 and 3 are provided for wild-type PAD2, 3 and 4, respectively. It should be understood that wild-type nucleic acid and amino acid sequences herein refer to those nucleic acid and amino acid sequences prevalent among a population and serve as a reference for their respective variants. As an example, SEQ ID NO:61 in Table 3 (GI number: 1519314340 and Accession number: NM_012387) identifies the wild-type nucleic acid sequence for hPAD4 while SEQ ID NO:62 (GI number: 216548487 and Accession number: NP_036519) identifies the wild-type amino acid sequence for hPAD4.

The sequence identifiers in Tables 1, 2 and 3 also are provided for variants of PAD2, 3 and 4 respectively. It should be understood that a variant refers to a nucleic acid sequence that is similar but different from the wild-type nucleic acid sequence.

A variant in any of the Tables below can include a nucleic acid substitution that can be the result of, for example, alternative splicing (e.g. splice variant). As an example, SEQ ID NO:69 in Table 3 (GI number: 767903519 and Accession number: XM_011541150.1:c.23G>A) is a hPAD4 nucleic acid splice variant of SEQ ID NO:61.

A variant in any of the Tables below can also include, for example, a nucleic acid substitution (e.g. SNP). As an example, SEQ ID NO:63 in Table 3 (GI number: 216548486 and Accession number: NM_012387.2:c.23G>A) is a hPAD4 nucleic acid variant of SEQ ID NO:61 and includes a single nucleic acid substitution at nucleic acid position 23, resulting in the substitution of G (guanosine) for A (adenine).

It should be understood that a variant also refers to an amino acid sequence that is similar but different to the wild-type amino acid sequence.

A variant in any of the Tables below can further include amino acid substitutions that can be the result of, for example, alternative splicing (e.g. splice variant). As an example, SEQ ID NO:70 in Table 3 (GI number: 767903520 and Accession number: XP_011539452.1:p.Arg8His), which is encoded by SEQ ID NO:69 described above, is a hPAD4 that includes an amino acid substitution at position 8, resulting in a substitution of Arg (arginine) for His (histidine).

A variant in any of the Tables below can include, for example, amino acid substitutions that can be the result of genetic inheritance (e.g. SNP). As an example, SEQ ID NO:64 in Table 3 (GI number: 216548487 and Accession number: NP_036519.2:p.Arg8His), which is encoded by SEQ ID NO:63 described above, is a hPAD4 that includes an amino acid substitution at position 8, resulting in a substitution of Arg (arginine) for His (histidine).

TABLE 1

| | | | GenBank |
|---|---|---|---|
| | SEQ | GI | Accession |
| Molecule Type | ID NO | Number | Number |
| Homo sapiens peptidyl | 1 | 1519245591 | NM_007365 |
| arginine deiminase 2 | | | |
| (PADI2), mRNA | | | |
| protein-arginine | 2 | 122939159 | NP_031391 |
| deiminase type-2 | | | |
| [Homo sapiens] | | | |
| PREDICTED: Homo sapiens | 3 | 1370451734 | XM_017000148 |
| peptidyl arginine | | | |
| deiminase 2 (PADI2), | | | |
| transcript variant | | | |
| X2, mRNA | | | |
| protein-arginine deiminase | 4 | 1034554998 | XP_016855637 |
| type-2 isoform X1 | | | |
| [Homo sapiens] | | | |

TABLE 2

| | | | GenBank | Amino Acid | |
|---|---|---|---|---|---|
| Molecule Type | SEQ ID NO | GI Number | Accession Number | [Codon] | SO Term |
| Homo sapiens peptidyl | 5 | 122939160 | NM_016233 | N/A | N/A |
| arginine deiminase 3 | | | | | |
| (PADI3), mRNA | | | | | |
| protein-arginine deiminase | 6 | 122939161 | NP_057317 | N/A | N/A |
| type-3 [Homo sapiens] | | | | | |
| PADI3 transcript | 7 | 122939160 | NM_016233.2: | I [ATC] > | Coding |
| | | | c.154A > G | V [GTC] | Sequence Variant |
| protein-arginine deiminase | 8 | 122939161 | NP_057317.2: | I (Ile) > | Missense Variant |
| type-3 | | | p.Ile52Val | V (Val) | |
| PADI3 transcript | 9 | 122939160 | NM_016233.2: | L [CTC] > | Coding |
| | | | c.335T > A | H [CAC] | Sequence Variant |
| protein-arginine deiminase | 10 | 122939161 | NP_057317.2: | L (Leu) > | Missense Variant |
| type-3 | | | p.Leu112His | H (His) | |
| PADI3 transcript | 11 | 122939160 | NM_016233.2: | V [GTG] > | Coding |
| | | | c.511G > A | M [ATG] | Sequence Variant |
| protein-arginine deiminase | 12 | 122939161 | NP_057317.2: | V (Val) > | Missense Variant |
| type-3 | | | p.Val171Met | M (Met) | |
| PADI3 transcript | 13 | 122939160 | NM_016233.2: | A [GCA] > | Coding |
| | | | c.881C > T | V [GTA] | Sequence Variant |
| protein-arginine deiminase | 14 | 122939161 | NP_057317.2: | A (Ala) > | Missense Variant |
| type-3 | | | p.Ala294Val | V (Val) | |
| PADI3 transcript | 15 | 122939160 | NM_016233.2: | A [GCC] > | Coding |
| | | | c.1744G > A | T [ACC] | Sequence Variant |
| protein-arginine deiminase | 16 | 122939161 | NP_057317.2: | A (Ala) > | Missense Variant |
| type-3 | | | p.Ala582Thr | T (Thr) | |
| PADI3 transcript | 17 | 122939160 | NM_016233.2: | P [CCC] > | Coding |
| | | | c.1813C > A | T [ACC] | Sequence Variant |
| protein-arginine deiminase | 18 | 122939161 | NP_057317.2: | P (Pro) > | Missense Variant |
| type-3 | | | p.Pro605Thr | T (Thr) | |
| PADI3 transcript | 19 | 122939160 | NM_016233.2: | R [CGG] > | Coding |
| | | | c.1853G > A | Q [CAG] | Sequence Variant |
| protein-arginine deiminase | 20 | 122939161 | NP_057317.2: | R (Arg) > | Missense Variant |
| type-3 | | | p.Arg618Gln | Q (Gln) | |
| Predicted: Homo sapiens | 21 | 1034559140 | XM_011541571 | N/A | N/A |
| peptidyl arginine deiminase | | | | | |
| 3 (PADI3), transcript | | | | | |
| variant X1, mRNA | | | | | |
| protein-arginine deiminase | 22 | 767904616 | XP_011539873 | N/A | N/A |
| type-3 isoform X1 [Homo | | | | | |
| sapiens] | | | | | |
| Predicted: PADI3 transcript | 23 | 1034559140 | XM_011541571.2: | I [ATC] > | Coding |
| variant X1 | | | c.40A > G | V [GTC] | Sequence Variant |
| | | | | I (Ile) > | |
| | | | | V (Val) | |
| protein-arginine deiminase | 24 | 767904616 | XP_011539873.1: | | Missense Variant |
| type-3 isoform X1 | | | p.Ile14Val | | |
| Predicted: PADI3 transcript | 25 | 1034559140 | XM_011541571.2: | L [CTC] > | Coding |
| variant X1 | | | c.221T > A | H [CAC] | Sequence Variant |
| protein-arginine deiminase | 26 | 767904616 | XP_011539873.1: | L (Leu) > | Missense Variant |
| type-3 isoform X1 | | | p.Leu74His | H (His) | |
| Predicted: PADI3 transcript | 27 | 1034559140 | XM_011541571.2: | V [GTG] > | Coding |
| variant X1 | | | c.397G > A | M [ATG] | Sequence Variant |
| protein-arginine deiminase | 28 | 767904616 | XP_011539873.1: | V (Val) > | Missense Variant |
| type-3 isoform X1 | | | p.Val133Met | M (Met) | |
| PADI3 transcript variant X1 | 29 | 1034559140 | XM_011541571.2: | A [GCA] > | Coding |
| | | | c.767C > T | V [GTA] | Sequence Variant |
| protein-arginine deiminase | 30 | 767904616 | XP_011539873.1: | A (Ala) > | Missense Variant |
| type-3 isoform X1 | | | p.Ala256Val | V (Val) | |
| PADI3 transcript variant X1 | 31 | 1034559140 | XM_011541571.2: | A [GCC] > | Coding |
| | | | c.1630G > A | T [ACC] | Sequence Variant |
| protein-arginine deiminase | 32 | 767904616 | XP_011539873.1: | A (Ala) > | Missense Variant |
| type-3 isoform X1 | | | p.Ala544Thr | T (Thr) | |
| PADI3 transcript variant X1 | 33 | 1034559140 | XM_011541571.2: | P [CCC] > | Coding |
| | | | c.1699C > A | T [ACC] | Sequence Variant |
| protein-arginine deiminase | 34 | 767904616 | XP_011539873.1: | P (Pro) > | Missense Variant |
| type-3 isoform X1 | | | p.Pro567Thr | T (Thr) | |
| PADI3 transcript variant X1 | 35 | 1034559140 | XM_011541571.2: | R [CGG] > | Coding |
| | | | c.1739G > A | Q [CAG] | Sequence Variant |
| protein-arginine deiminase | 36 | 767904616 | XP_011539873.1: | R (Arg) > | Missense Variant |
| type-3 isoform X1 | | | p.Arg580Gln | Q (Gln) | |
| Homo sapiens peptidyl | 37 | 1034559141 | XM_017001463 | N/A | N/A |
| arginine deiminase 3 | | | | | |
| (PADI3), transcript variant | | | | | |
| X2, mRNA | | | | | |
| protein-arginine deiminase | 38 | 1034559142 | XP_016856952 | N/A | N/A |
| type-3 isoform X2 [Homo | | | | | |
| sapiens] | | | | | |
| PADI3 transcript variant X2 | 39 | 1034559141 | XM_017001463.1: | N/A | Genic Upstream |
| | | | c | | Transcript Variant |
| PADI3 transcript variant X2 | 40 | 1034559141 | XM_017001463.1: | N/A | 5 Prime UTR |
| | | | c | | Variant |
| PADI3 transcript variant X2 | 41 | 1034559141 | XM_017001463.1: | A [GCA] > | Coding |
| | | | c.344C > T | V [GTA] | Sequence Variant |
| protein-arginine deiminase | 42 | 1034559142 | XP_016856952.1: | A (Ala) > | Missense Variant |
| type-3 isoform X2 | | | p.Ala115Val | V (Val) | |
| PADI3 transcript variant X2 | 43 | 1034559141 | XM_017001463.1: | A [GCC] > | Coding |
| | | | c.1207G > A | T [ACC] | Sequence Variant |
| protein-arginine deiminase | 44 | 1034559142 | XP_016856952.1: | A (Ala) > | Missense Variant |
| type-3 isoform X2 | | | p.Ala403Thr | T (Thr) | |
| PADI3 transcript variant X2 | 45 | 1034559141 | XM_017001463.1: | P [CCC] > | Coding |
| | | | c.1276C > A | T [ACC] | Sequence Variant |
| protein-arginine deiminase | 46 | 1034559142 | XP_016856952.1: | P (Pro) > | Missense Variant |
| type-3 isoform X2 | | | p.Pro426Thr | T (Thr) | |
| PADI3 transcript variant X2 | 47 | 1034559141 | XM_017001463.1: | R [CGG] > | Coding |
| | | | c.1316G > A | Q [CAG] | Sequence Variant |
| protein-arginine deiminase | 48 | 1034559142 | XP_016856952.1: | R (Arg) > | Missense Variant |
| type-3 isoform X2 | | | p.Arg439Gln | Q (Gln) | |
| Homo sapiens peptidyl | 49 | 1034559143 | XM_011541572 | N/A | N/A |
| arginine deiminase 3 | | | | | |
| (PADI3), transcript variant | | | | | |
| X3, mRNA | | | | | |
| protein-arginine deiminase | 50 | 767904618 | XP_011539874 | N/A | N/A |
| type-3 isoform X3 [Homo | | | | | |
| sapiens] | | | | | |
| PADI3 transcript variant X3 | 51 | 1034559143 | XM_011541572.2: | I [ATC] > | Coding |
| | | | c.154A > G | V [GTC] | Sequence Variant |
| protein-arginine deiminase | 52 | 767904618 | XP_011539874.1: | I (Ile) > | Missense Variant |
| type-3 isoform X3 | | | p.Ile52Val | V (Val) | |
| PADI3 transcript variant X3 | 53 | 1034559143 | XM_011541572.2: | L [CTC] > | Coding |
| | | | c.335T > A | H [CAC] | Sequence Variant |
| protein-arginine deiminase | 54 | 767904618 | XP_011539874.1: | L (Leu) > | Missense Variant |
| type-3 isoform X3 | | | p.Leu112His | H (His) | |
| PADI3 transcript variant X3 | 55 | 1034559143 | XM_011541572.2: | V [GTG] > | Coding |
| | | | c.511G > A | M [ATG] | Sequence Variant |
| protein-arginine deiminase | 56 | 767904618 | XP_011539874.1: | V (Val) > | Missense Variant |
| type-3 isoform X3 | | | p.Val171Met | M (Met) | |
| PADI3 transcript variant X3 | 57 | 1034559143 | XM_011541572.2: | A [GCA] > | Coding |
| | | | c.881C > T | V [GTA] | Sequence Variant |
| protein-arginine deiminase | 58 | 767904618 | XP_011539874.1: | A (Ala) > | Missense Variant |
| type-3 isoform X3 | | | p.Ala294Val | V (Val) | |
| PADI3 transcript variant X3 | 59 | 1034559143 | XM_011541572.2: | N/A | Genic Downstream |
| | | | c. | | Transcript Variant |

TABLE 3

| | | | GenBank | Amino Acid | |
|---|---|---|---|---|---|
| Molecule Type | SEQ ID NO | GI Number | Accession Number | [Codon] | SO Term |
| Homo sapiens peptidyl | 61 | 1519314340 | NM_012387 | N/A | N/A |
| arginine deiminase 4 | | | | | |
| (PADI4), mRNA | | | | | |
| protein-arginine | 62 | 216548487 | NP_036519 | N/A | N/A |
| deiminase type-4 [Homo | | | | | |
| sapiens] | | | | | |
| PADI4 transcript | 63 | 216548486 | NM_012387.2: | R [CGT] > | Coding |
| | | | c.23G > A | H [CAT] | Sequence Variant |
| protein-arginine | 64 | 216548487 | NP_036519.2: | R (Arg) > | Missense Variant |
| deiminase type-4 | | | p.Arg8His | H (His) | |
| PADI4 transcript | 65 | 216548486 | NM_012387.2: | R [CGT] > | Coding |
| | | | c.23G > T | L [CTT] | Sequence Variant |
| protein-arginine | 66 | 216548487 | NP_036519.2: | R (Arg) > | Missense Variant |
| deiminase type-4 | | | p.Arg8Leu | L (Leu) | |
| PADI4 transcript variant X3 | 67 | 767903523 | XM_011541152.1: | N/A | Genic Upstream |
| | | | c. | | Transcript Variant |
| PADI4 transcript variant X8 | 68 | 767903533 | XM_011541157.1: | N/A | Genic Upstream |
| | | | c. | | Transcript Variant |
| PADI4 transcript variant X1 | 69 | 767903519 | XM_011541150.1: | R [CGT] > | Coding |
| | | | c.23G > A | H [CAT] | Sequence |
| | | | | | Variant |
| protein-arginine | 70 | 767903520 | XP_011539452.1: | R (Arg) > | Missense |
| deiminase type-4 isoform X1 | | | p.Arg8His | H (His) | Variant |
| PADI4 transcript variant X1 | 71 | 767903519 | XM_011541150.1: | R [CGT] > | Coding |
| | | | c.23G > T | L [CTT] | Sequence |
| | | | | | Variant |
| protein-arginine | 72 | 767903520 | XP_011539452.1: | R (Arg) > | Missense |
| deiminase type-4 isoform X1 | | | p.Arg8Leu | L (Leu) | Variant |
| PADI4 transcript variant X2 | 73 | 767903521 | XM_011541151.1: | R [CGT] > | Coding |
| | | | c.23G > A | H [CAT] | Sequence |
| | | | | | Variant |
| protein-arginine | 74 | 767903522 | XP_011539453.1: | R (Arg) > | Missense |
| deiminase type-4 isoform X2 | | | p.Arg8His | H (His) | Variant |
| PADI4 transcript variant X2 | 75 | 767903521 | XM_011541151.1: | R [CGT] > | Coding |
| | | | c.23G > T | L [CTT] | Sequence |
| | | | | | Variant |
| protein-arginine | 76 | 767903522 | XP_011539453.1: | R(Arg)> | Missense |
| deiminase type-4 isoform X2 | | | p.Arg8Leu | L (Leu) | Variant |
| PADI4 transcript variant X4 | 77 | 767903525 | XM_011541153.1: | R [CGT] > | Coding |
| | | | c.23G > A | H [CAT] | Sequence |
| | | | | | Variant |
| protein-arginine | 78 | 767903526 | XP_011539455.1: | R (Arg) > | Missense |
| deiminase type-4 isoform X4 | | | p.Arg8His | H (His) | Variant |
| PADI4 transcript variant X4 | 79 | 767903525 | XM_0115411 | R [CGT] > | Coding |
| | | | 53.1:c.23G>T | L [CTT] | Sequence |
| | | | | | Variant |
| protein-arginine | 80 | 767903526 | XP_011539455.1: | R (Arg) > | Missense |
| deiminase type-4 isoform X4 | | | p.Arg8Leu | L (Leu) | Variant |
| PADI4 transcript variant X6 | 81 | 767903529 | XM_011541155.1: | R [CGT] > | Coding |
| | | | c.23G > A | H [CAT] | Sequence |
| | | | | | Variant |
| protein-arginine | 82 | 767903530 | XP_011539457.1: | R (Arg) > | Missense |
| deiminase type-4 isoform X5 | | | p.Arg8His | H (His) | Variant |
| PADI4 transcript variant X6 | 83 | 767903529 | XM_011541155.1: | R [CGT] > | Coding |
| | | | c.23G > T | L [CTT] | Sequence |
| | | | | | Variant |
| protein-arginine | 84 | 767903530 | XP_011539457.1: | R (Arg) > | Missense |
| deiminase type-4 isoform X5 | | | pArg8Leu | L (Leu) | Variant |
| PADI4 transcript variant X7 | 85 | 767903531 | XM_011541156.1: | R [CGT] > | Coding |
| | | | c.23G > A | H [CAT] | Sequence |
| | | | | | Variant |
| protein-arginine | 86 | 767903532 | XP_011539458.1: | R (Arg) > | Missense |
| deiminase type-4 isoform X6 | | | p.Arg8His | H (His) | Variant |
| PADI4 transcript variant X7 | 87 | 767903531 | XM_011541156.1: | R [CGT] > | Coding |
| | | | c.23G > T | L [CTT] | Sequence |
| | | | | | Variant |
| protein-arginine | 88 | 767903532 | XP_011539458.1: | R (Arg) > | Missense |
| deiminase type-4 isoform X6 | | | p.Arg8Leu | L (Leu) | Variant |
| PADI4 transcript variant X5 | 89 | 1034557308 | XM_011541154.2: | R [CGT] > | Coding |
| | | | c.23G > A | H [CAT] | Sequence |
| | | | | | Variant |
| protein-arginine | 90 | 767903528 | XP_011539456.1: | R (Arg) > | Missense |
| deiminase type-4 isoform X4 | | | p.Arg8His | H (His) | Variant |
| PADI4 transcript variant X5 | 91 | 1034557308 | XM_011541154.2: | R [CGT] > | Coding |
| | | | c.23G > T | L [CTT] | Sequence |
| | | | | | Variant |
| protein-arginine | 92 | 767903528 | XP_011539456.1: | R (Arg) > | Missense |
| deiminase type-4 isoform X4 | | | p.Arg8Leu | L (Leu) | Variant |

It should be noted that “polypeptide” includes a short oligopeptide having between 2 and 30 amino acids (e.g., 2, 3, 4, 5, 6, 7, 8, 9, 10, 12, 14, 16, 18, 20, 25 or 30 amino acids) as well as longer amino acid chains, e.g., more than 30 amino acids, more than 50 amino acids, more than 100 amino acids, more than 150 amino acids, more than 200 amino acids, more than 300 amino acids, more than 400 amino acids, more than 500 amino acids, or more than 600 amino acids. In some embodiments, the PAD can be a full-length, wild-type polypeptide. PAD polypeptides can include unnatural amino acids not encoded by the natural genetic code.

In some embodiments, the purified polypeptide includes a PAD antigenic fragment. In some embodiments, a PAD antigenic fragment includes more than 3, more than 5, more than 10, more than 15, more than 20, more than 25, more than 50, more than 75, more than 100, more than 125, more than 150, more than 200, more than 250, more than 300, more than 350, more than 400, more than 500, or more than 600 consecutive amino acids of a full-length PAD polypeptide. In some embodiments, a PAD antigenic fragment includes less than 100%, less than 95%, less than 90%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of consecutive amino acids of full-length PAD. In some embodiments, a PAD antigenic fragment is a PAD peptide fragment.

In some embodiments, a PAD or antigenic fragment thereof can be a mammalian PAD. In some embodiments, a PAD or antigenic fragment thereof can be human. In some embodiments, a PAD or antigenic fragment thereof can be a PAD or antigenic fragment thereof of one of the organisms of the present disclosure. In some embodiments, a PAD or antigenic fragment thereof can include any of the variants or single nucleotide polymorphisms in Tables 1-3.

The present disclosure provides a method of diagnosing RA. The method includes: (a) contacting a biological sample from a subject suspected of having RA with at least one peptidyl arginine deiminase (PAD) protein or an antigenic fragment thereof, and (b) detecting the presence of an autoantibody bound to the at least one PAD protein or an antigenic fragment thereof, wherein the presence of said autoantibody is indicative of RA, wherein the at least one PAD protein comprises PAD1 or an antigenic fragment thereof. In certain embodiments, the at least one PAD protein comprises PAD1 or an antigenic fragment thereof and another PAD protein, such as PAD4, or an antigenic fragment thereof. However, it is understood that other combinations of PAD proteins or an antigenic fragment thereof can be useful for the presents disclosure and it is not limited to PAD1 and PAD4.

As provided herein, the present disclosure has identified for the first time that PAD1 is a novel autoantigen in RA, and that detection of autoantibodies against PAD1 or an antigenic fragment thereof can discriminate between RA and non-RA patients with high sensitivity. Accordingly, in some embodiments, the method of diagnosing RA involves at least one PAD protein that is PAD1 or an antigenic fragment thereof.

The present disclosure is also based in part on the finding that PAD1 or an antigenic fragment thereof can be combined with one or more additional PAD protein or antigenic fragment thereof for the diagnosis of RA. For example, PAD1 or an antigenic fragment thereof can be combined with one or more additional PAD protein or antigenic fragment thereof for detecting autoantibodies against PAD1 or an antigenic fragment thereof, as well as autoantibodies against the one or more additional PAD protein or antigenic fragment thereof. Accordingly, in some embodiments, the method of diagnosing RA involves PAD1 or an antigenic fragment thereof and one additional PAD protein or an antigenic fragment thereof. In other embodiments, the method of diagnosing RA involves PAD1 or an antigenic fragment thereof and two additional PAD proteins or an antigenic fragment thereof. In yet further embodiments, the method of diagnosing RA involves PAD1 or an antigenic fragment thereof and three additional PAD proteins or an antigenic fragment thereof. In even further embodiments, the method of diagnosing RA involves PAD1 or an antigenic fragment thereof and all four additional PAD proteins or an antigenic fragment thereof.

In particular, as provided herein, the combination of PAD1 and PAD4 or an antigenic fragment thereof is able to discriminate between RA and non-RA patients with high sensitivity. Accordingly, in certain embodiments the method of diagnosing RA involves PAD1 and PAD4 or an antigenic fragment thereof. However, it is further understood that the method of diagnosing RA can also involve PAD1 or an antigenic fragment thereof combined with any of PAD2, PAD3, and PAD6 or an antigenic fragment thereof. For example, in addition to the combination of PAD1 and PAD4 or an antigenic fragment thereof, PAD1 or an antigenic fragment thereof can also be individually combined with PAD2, PAD3, or PAD6 or an antigenic fragment thereof. PAD1 or an antigenic fragment thereof can also be combined with each of PAD2 and PAD3, PAD2 and PAD6, or PAD3 and PAD6 or an antigenic fragment thereof. Furthermore, PAD1 or an antigenic fragment thereof can also be combined with PAD2, PAD3, and PAD6 or an antigenic fragment thereof.

In other embodiments, the method of diagnosing RA can involve PAD1 and PAD4 or an antigenic fragment thereof combined with any of PAD2, PAD3, and PAD6 or an antigenic fragment thereof. For example, in some embodiments, PAD1 and PAD4 or an antigenic fragment thereof are combined with PAD3 or an antigenic fragment thereof. In other embodiments, PAD1 and PAD4 or an antigenic fragment thereof are combined with PAD2 or an antigenic fragment thereof. In yet further embodiments, PAD1 and PAD4 or an antigenic fragment thereof are combined with PAD6 or an antigenic fragment thereof. In some embodiments, PAD1 and PAD4 or an antigenic fragment thereof are combined with PAD3 and PAD2 or an antigenic fragment thereof. In other embodiments, PAD1 and PAD4 or an antigenic fragment thereof are combined with PAD3 and PAD6 or an antigenic fragment thereof. In still other embodiments, PAD1 and PAD4 or an antigenic fragment thereof are combined with PAD2 and PAD6 or an antigenic fragment thereof. In yet other embodiments, PAD1 and PAD4 or an antigenic fragment thereof are combined with PAD2, PAD3, and PAD6 or an antigenic fragment thereof.

As provided herein, the at least one PAD protein or antigenic fragment thereof described above can be contacted with a biological sample, and the presence of an autoantibody reactive with the at least one PAD protein or an antigenic fragment thereof can be detected. The specificity of the PAD proteins described herein are sensitive enough that if autoantibodies are detected in the biological sample, the biological sample donor can be considered to have RA. By way of example, and without limitation, PAD1 or an antigenic fragment thereof can be used and anti-PAD1 autoantibodies can be detected according to the methods described herein. Similarly, another exemplary embodiment can involve PAD1 and PAD4 or an antigenic fragment thereof and if autoantibodies are detected according to the methods described herein, the biological sample donor can be considered to have RA.

In certain embodiments, the PAD autoantibody that is detected is a specific isotype. For example, in some embodiments the PAD autoantibody that is detected is an IgA isotype (e.g., anti-PAD1 IgA or anti-PAD4 IgA). In other the autoantibody that is detected is an IgG isotype (e.g., anti-PAD1 IgG or anti-PAD4 IgG). In specific embodiments, the autoantibody isotype is anti-PAD1 IgA. In other embodiments, the autoantibody isotype is anti-PAD4 IgG. In still further embodiments, the autoantibody isotype is anti-PAD4 IgA. In certain embodiments both detection of IgG and IgA isotypes are combined to increase the detection of autoantibodies. Accordingly, it is understood that throughout the present disclosure reference to an anti-PAD1 or anti-PAD4 autoantibody without recitation of a specific isotype can include specific isotypes, such as IgA and IgG.

In some aspects of the present invention, where two or more PAD proteins or an antigenic fragment thereof are used, the present disclosure provides the option to be able to identify which autoantibody is detected. For example, in some embodiments, PAD1 and PAD4 or an antigenic fragment thereof are used and the detection of an autoantibody can be traced to reacting with PAD1 or PAD4 or an antigenic fragment thereof. Alternatively, PAD1, PAD4, and PAD3 or an antigenic fragment thereof are used and the detection of an autoantibody can be traced to reacting with PAD1, PAD4, or PAD3 or an antigenic fragment thereof. It should be noted that the examples provided above are understood that be merely exemplary and that other combinations of two or more PAD proteins or an antigenic fragment thereof can be used according to the present disclosure.

Where two or more PAD proteins or an antigenic fragment thereof are used, and an autoantibody is detected, it may be desired to determine what PAD proteins or an antigenic fragment thereof was reactive with the autoantibody. Various techniques and assay designs for associating the detection of an autoantibody with its autoantigenic substrate are known in the art. For example, one illustrative approach includes spatial separation of the two or more PAD proteins or antigenic fragments thereof such that each of the two or more PAD proteins or antigenic fragments occupies a unique space on a solid support. Another exemplary approach includes temporal separation such that each of the two or more PAD proteins or antigenic fragments is assayed sequentially. While these examples are illustrative, they are not intended to be exhaustive of all the designs available, and it is understood that embodiments of the present disclosure involving two or more PAD proteins or an antigenic fragment thereof can be used in any technique known in the art suitable for multiplexing.

As provided herein, the detection of two or more different autoantibodies that are each reactive to a specific PAD protein or antigenic fragment thereof can improve the ability to diagnose RA, as compared to detection of the individual autoantibody alone. For example, as disclosed herein, detection of anti-PAD1 and anti-PAD4 was able to discriminate RA from non-RA patients better than anti-PAD1 or anti-PAD4 alone. Thus, combining the detection of two or more different autoantibodies can improve RA diagnosis.

In some aspects of the present disclosure, when two or more different autoantibodies are detected a composite score is created. For example, in some embodiments, a composite score can be generated by combining the two or more different autoantibodies that are detected. A composite score can also be created by including a negative association of one of the PAD proteins or antigenic fragments thereof, such as for example, using the following exemplary equation=(anti-PAD1+anti-PAD4)/(anti-PAD6). The creation of a composite score can also involve generating an artificial intelligence (AI) based score.

In some aspects, detection of an autoantibody that is reactive with the at least one PAD protein or antigenic fragment thereof includes quantifying the presence of the autoantibody. However, it is also understood that in some applications of the present disclosure, a qualitative assay may be desired and that quantification is not necessary. For example, in some embodiments, detecting an autoantibody against the at least one PAD protein or antigenic fragment thereof is sufficient to diagnose RA.

Also provided herein is a method of monitoring the progression of rheumatoid arthritis (RA), that includes (a) contacting a biological sample from a subject having or suspected of having RA with at least one peptidyl arginine deiminase (PAD) protein or an antigenic fragment thereof, and (b) detecting the presence of an autoantibody reactive with the at least one PAD protein or an antigenic fragment thereof, wherein the presence of said autoantibody is indicative of disease progression, wherein the at least one PAD protein comprises PAD1, or PAD1 and PAD4.

For example, in certain embodiments an increase in the amount of an anti-PAD autoantibody, such as for example an anti-PAD autoantibody that includes anti-PAD1 or anti-PAD1 and anti-PAD4, can indicate disease progression. In other embodiments, a change in the ratio between two or more anti-PAD1 antibodies can indicate disease progression. A change in the amount of the anti-PAD autoantibody can be relative to a previous biological sample obtained from the same subject, or relative to a reference standard.

Accordingly, in some embodiments the method of monitoring the progression of rheumatoid arthritis (RA) includes contacting a biological sample from a subject having or suspected of having RA with PAD1 or an antigenic fragment thereof and detecting the presence of anti-PAD1. In other embodiments, the method of monitoring the progression of rheumatoid arthritis (RA) includes contacting a biological sample from a subject having or suspected of having RA with PAD1 and PAD4 or an antigenic fragment thereof and detecting the presence of anti-PAD1 and/or anti-PAD4.

The present disclosure also demonstrates that the detection of anti-PAD3 also correlates with anti-PAD1 and anti-PAD4 in the biological samples from RA patients. Therefore, in some embodiments, the method of monitoring the progression of rheumatoid arthritis (RA) includes contacting a biological sample from a subject having or suspected of having RA with PAD1 and PAD3 or an antigenic fragment thereof and detecting the presence of anti-PAD1 and/or anti-PAD3. Additionally, in some embodiments, the method of monitoring the progression of rheumatoid arthritis (RA) includes contacting a biological sample from a subject having or suspected of having RA with PAD1, PAD4, and PAD3 or an antigenic fragment thereof and detecting the presence of anti-PAD1, anti-PAD4, and/or anti-PAD3.

RA stage is commonly characterized into four main stages, categorized by clinical and radiologic criteria. Stage I (early RA) generally involves no destructive changes observed upon radiographic examination, and may involve initial inflammation in the joint capsule and swelling of synovial tissue. Stage II (moderate progression) generally involves radiographic evidence of periarticular osteoporosis, with or without slight subchondral bone destruction; slight cartilage destruction is possible; joint mobility is possibly limited, but no joint deformities are observed; adjacent muscle atrophy is present; extra-articular soft tissue lesions (e.g., nodules and tenosynovitis) are possible. Stage III (severe progression) generally involves radiographic evidence of cartilage and bone destruction in addition to periarticular osteoporosis; joint deformity (e.g., subluxation, ulnar deviation, or hyperextension) without fibrous or bony ankylosis; muscle atrophy is extensive; extra-articular soft tissue lesions (e.g., nodules, tenosynovitis) are possible. Stage IV (terminal progression) generally involves the presence of fibrous or bony ankylosis, along with criteria of stage III.

As described above, anti-PAD4 is generally associated with severe RA, and it often present in RA patients with joint erosion. Thus, in some embodiments detection of anti-PAD1 is indicative of moderate to severe stage RA. In some embodiments detection of anti-PAD1 and anti-PAD4 is indicative of moderate to severe stage RA. In some embodiments, detection of anti-PAD1 and anti-PAD3 is indicative of moderate to severe stage RA. In some embodiments, detection of anti-PAD1, anti-PAD4, and anti-PAD3 is indicative of moderate to severe stage RA. In specific embodiments, moderate to severe stage RA comprises the presence of joint erosion.

It is also known that the detection pattern of anti-PAD2 is generally not associated with anti-PAD3 or anti-PAD4, and is inversely correlated with progressive joint damage. Therefore, in some embodiments, detection of anti-PAD2 is indicative of RA that is not moderate to severe stage RA.

The present disclosure has also found that anti-PAD2 and anti-PAD6 correlate in the biological samples from RA patients. Accordingly, in some embodiments, detection of anti-anti-PAD6 is indicative of RA that is not moderate to severe stage RA. In other embodiments, detection of anti-PAD2 and anti-PAD6 is indicative of RA that is not moderate to severe stage RA.

In other aspects of the present disclosure, the method of monitoring disease progression involves (a) contacting a biological sample from a subject having RA with at least one peptidyl arginine deiminase (PAD) protein or an antigenic fragment thereof, and (b) detecting the absence of an autoantibody reactive with the at least one PAD protein or an antigenic fragment thereof, wherein the absence of said autoantibody is indicative of disease progression, wherein the at least one PAD protein comprises PAD1, or PAD1 and PAD4.

For example, in certain embodiments the absence in the amount of an anti-PAD autoantibody, such as for example an anti-PAD autoantibody that includes anti-PAD1 or anti-PAD1 and anti-PAD4, can indicate a decrease or no change in disease progression. In certain embodiments, a decrease or no change in the amount of the anti-PAD autoantibody can be relative to a previous biological sample obtained from the same subject, or relative to a reference standard.

As described above, the presence of anti-PAD4 is often found in RA patients with a more severe form of RA, and detection of anti-PAD4 strongly correlates with anti-PAD1, as well as anti-PAD3. Therefore, in patients known to have RA, the absence of anti-PAD4, anti-PAD1, and/or anti-PAD3 can indicate a less severe form of RA. The absence of anti-PAD4, anti-PAD1, and/or anti-PAD3 may be even more informative where the presence of anti-PAD2 and/or anti-PAD6 is detected. Conversely, the presence of anti-PAD1, anti-PAD4, and/or anti-PAD3, and the absence of anti-PAD2 and/anti-PAD6 may be informative for RA.

Accordingly, in some embodiments the method of monitoring the progression of rheumatoid arthritis (RA) includes contacting a biological sample from a subject having RA with PAD1 or an antigenic fragment thereof and detecting the absence of anti-PAD1. In other embodiments, the method of monitoring the progression of rheumatoid arthritis (RA) includes contacting a biological sample from a subject having RA with PAD1 and PAD4 or an antigenic fragment thereof and detecting the absence of anti-PAD1 and/or anti-PAD4. In some embodiments, the method of monitoring the progression of rheumatoid arthritis (RA) includes contacting a biological sample from a subject having RA with PAD1 and PAD3 or an antigenic fragment thereof and detecting the presence of anti-PAD1 and/or anti-PAD3. Additionally, in some embodiments, the method of monitoring the progression of rheumatoid arthritis (RA) can include contacting a biological sample from a subject having or suspected of having RA with PAD1, PAD4, and PAD3 or an antigenic fragment thereof and detecting the absence of anti-PAD1, anti-PAD4, and/or anti-PAD3. In specific embodiments, the absence of said autoantibody is indicative of early stage RA. In certain embodiments, early stage RA comprises little to no damage to the joints.

In addition, the presence of increased anti-PAD that includes, for example, anti-PAD1 or anti-PAD1 and anti-PAD4, in a subject compared to a healthy control individual can be indicative of the presence of RA, or the risk of developing RA. Accordingly, a measurable increase in an autoantibody to PAD, such as anti-PAD1 or anti-PAD1 and anti-PAD4, can be used to diagnose RA. Exemplary methods for detection and comparison of anti-PAD, such as anti-PAD1 or anti-PAD1 and anti-PAD4, levels to a control are provided herein and described further below.

In some embodiments, detection of an increased level of anti-PAD, such as anti-PAD1 or anti-PAD1 and anti-PAD4, compared to a healthy control individual is indicative of a subject having RA. In some embodiments, following diagnosis of RA using the compositions and methods provided herein, the presence of RA can be further corroborated based on a variety of symptoms associated with the onset or presence of RA.

Clinical symptoms associated with RA include, for example, pain and swelling of small and large bilateral joints, palindromic onset, monoarticular presentation, and extra-articular synovitis, like tenosynovitis and bursitis, polymyalgic-like onset and other symptoms including malaise, weight loss, fatigue, fever and disability. Grassi et al., Eur. J. Radiol., Suppl 1:S 18-24 (1998); Aletaha and Smolen, JAMA, 320(13):1360-1372 (2018).

In some embodiments, detection of an increased level of anti-PAD that includes, for example, anti-PAD1 or anti-PAD1 and anti-PAD4, in a subject compared to a healthy control is indicative of having severe RA. In other embodiments, detection of an increased level of anti-PAD that includes, for example, anti-PAD1 or anti-PAD1 and anti-PAD4, in a subject compared to an RA subject without an increased level of the same anti-PAD, is indicative of having severe RA. In some embodiments, having severe RA is considered by the degree of joint erosion or the risk of radiographic progression as determined by methods in the art. Detection of an increased level of anti-PAD that includes, for example, anti-PAD1 or anti-PAD1 and anti-PAD4, in a subject compared to a healthy control or compared to an RA subject without an increased level of anti-PAD that includes, for example, anti-PAD1 or anti-PAD1 and anti-PAD4, is indicative that the subject has a higher probability of having more progressed RA wherein joint erosion is severe. In some embodiments, a subject having increased anti-PAD that includes, for example, anti-PAD1 or anti-PAD1 and anti-PAD4, can be more than 5%, more than 10%, more than 15%, more than 20%, more than 25%, more than 30%, more than 35%, more than 40%, more than 45%, more than 50%, more than 60%, more than 70%, more than 80% or more than 90% likely to have more progressed RA where severe joint erosion is present. In other embodiments, a subject having increased anti-PAD can be more than 2-fold, more than 3-fold, more than 4-fold, more than 5-fold, more than 6-fold, more than 7-fold, more than 8-fold, more than 9-fold, or more than 10-fold likely to have more progressed RA where severe joint erosion is present.

In severe RA, joint erosion occurs when there is loss of bone and cartilage in the joint. Severity of joint erosion can be determined by, for example, the Sharp score method. See Sharp, Arthritis Rheum., 32:221-229 (1989); Brower, Arthritis Rheum., 33:316-324 (1990). The Sharp score assesses joints for narrowness and erosions, based upon radiographic images. Erosion scores range from 0-3.5 and joint space narrowing scores range from 0-4. A score of 0 indicates a normal joint with no narrowing or erosions and a score of 3.5-4 indicates an abnormal joint with erosions and narrowing. In some embodiments of the present disclosure, joint erosion in a subject is determined by use of the Sharp score.

In other embodiments, having severe RA is determined by the Health Assessment Questionnaire (HAQ) Disability Index (DI). Fries et al., Arthritis Rheum, 23(2):137-145 (1980); Bruce and Fries, Health Qual Life Outcomes, 1(1):20 (2003). The HAQ assesses physical ability in 8 sections including dressing, arising, eating, walking, hygiene, reach, grip and activities. Performing each session is allotted a score ranging from 0 (without any difficulty) to 3 (unable to do). The scores of the 8 sections are summed and divided by 8 to produce the DI. The DI, which ranges from 0 to 3, predicts disability, with a person able to complete a task without any difficulty (DI of 0), with some difficulty (DI of 1), with much difficulty (DI of 2), or unable to do (DI of 3).

In some embodiments, detection of an increased level of anti-PAD that includes, for example, anti-PAD1 or anti-PAD1 and anti-PAD4, compared to a healthy control individual indicates that the subject is at risk of developing clinical symptoms of RA. In some embodiments, a subject can be at risk of developing clinical symptoms of RA within less than 3 months, less than 6 months, less than 9 months, less than 12 months, less than 18 months, less than 2 years, less than 3 years, less than 4 years, less than 5 years, less than 6 years, less than 7 years, less than 8 years, less than 9 years, less than 10 years, less than 12 years, less than 14 years, or less than 16 years from the determination of the increased anti-PAD that includes, for example, anti-PAD1 or anti-PAD1 and anti-PAD4.

In some embodiments, the presence of an increased level of anti-PAD that includes, for example, anti-PAD1 or anti-PAD1 and anti-PAD4, compared to a healthy control individual indicates that the subject is more than 5%, more than 10%, more than 15%, more than 20%, more than 25%, more than 30%, more than 35%, more than 40%, more than 45%, more than 50%, more than 60%, more than 70%, or more than 80% or more than 90% likely to develop clinical symptoms of RA within 5 years following the determination of increased anti-PAD. In some embodiments, the presence of an increased level of anti-PAD that includes, for example, anti-PAD1 or anti-PAD1 and anti-PAD4, can indicate that the subject is more than 2-fold, more than 3-fold, more than 4-fold, more than 5-fold, more than 6-fold, more than 7-fold, more than 8-fold, more than 9-fold, or more than 10-fold likely to develop clinical symptoms of RA within 5 years following determination of the increased anti-PAD level compared to a healthy control individual.

Anti-PAD, such as, for example, anti-PAD1 or anti-PAD1 and anti-PAD4, can be detected in a variety of different biological samples obtained from a subject. Such samples include, for example, solid tissue and biological fluids. As used herein, the term “biological sample” refers to any specimen from the body of an organism that can be used for analysis or diagnosis. In the context of the present disclosure, a biological sample obtained from a subject can be any sample that contains or is suspected to contain autoantibodies and encompasses any material in which an anti-PAD autoantibody can be detected. For example, a biological sample can include a liquid sample such as whole blood, plasma, serum, synovial fluid, amniotic fluid, sputum, pleural fluid, peritoneal fluid, central spinal fluid, urine, saliva, tears or other body fluid that contains autoantibodies. A biological sample can also include a solid tissue sample such as bone marrow, tissue, buccal or other solid or semi-solid aggregate of cells.

In some embodiments, anti-PAD that includes, for example, anti-PAD1 or anti-PAD1 and anti-PAD4, is detected in whole blood, plasma, serum, synovial fluid or sputum. In some embodiments of the present disclosure, the level of the anti-PAD is detected. In other embodiments, anti-PAD-PAD complex can be formed using the compositions and methods described herein and an anti-PAD in the complex can be detected. Accordingly, the disclosure provides compositions that include an anti-PAD-PAD complex.

The biological samples of this disclosure can be obtained from any organism including, for example, mammals such as humans, primates such as monkeys, chimpanzees, orangutans and gorillas, cats, dogs, rabbits, farm animals such as cows, horses, goats, sheep and pigs, and rodents such as mice, rats, hamsters and guinea pigs.

In some embodiments, the biological sample can be a plurality of samples. In some embodiments the plurality of samples can be obtained periodically over the course of more than 12 hours, more than 1 day, more than 2 days, more than 3 days, more than 4 days, more than 5 days, more than 6 days, more than 7 days, more than 10 days, more than 14 days, more than 3 weeks, more than 1 month, more than 2 months, more than 3 months, more than 4 months, more than 5 months, more than 6 months, more than 9 months, more than 12 months, more than 18 months, more than 24 months, more than 30 months, more than 3 years months, more than 4 years or more than 5 years.

In some embodiments, the samples of the present disclosure can be collected and processed fresh. In other embodiments, the samples of the present disclosure can be frozen, stored and processed at a later date.

In some embodiments, the present disclosure provides a method of determining the level of anti-PAD, that includes, for example, anti-PAD1 or anti-PAD1 and anti-PAD4, in a subject to determine if that subject has RA, severe RA or joint erosion, including severe joint erosion. It is noted that, as used herein, the terms “subject,” “organism,” “individual” or “patient” are used as synonyms and interchangeably, and refer to a vertebrate mammal. Mammals include humans, primates such as monkeys, chimpanzees, orangutans and gorillas, cats, dogs, rabbits, farm animals such as cows, horses, goats, sheep and pigs, and rodents such as mice, rats, hamsters and guinea pigs. The subjects of this disclosure can include healthy subjects, asymptomatic subjects, and diseased subjects.

In some embodiments, the diseased subjects can suffer from any disease associated with aberrant anti-PAD levels that includes, for example, anti-PAD1 levels or anti-PAD1 and anti-PAD4 levels. It is noted that the term “aberrant anti-PAD levels” refers to anti-PAD levels, such as anti-PAD1 levels or anti-PAD1 and anti-PAD4 levels, in a sample that measurably deviate from the median anti-PAD levels found in a population of healthy subjects. In some embodiments, the aberrant anti-PAD levels can be higher than the median anti-PAD levels. In some embodiments, the aberrant anti-PAD levels can be lower than the median anti-PAD levels.

In some embodiments, the healthy subjects can have never suffered from a certain disease. In some embodiments, the healthy subjects can be previously diseased. In some embodiments, the healthy subjects can be undergoing a routine medical checkup. In some embodiments, the healthy subjects can be members of a control group in, for example, a clinical trial. In some embodiments, the healthy subjects can be at risk of contracting a disease, as determined by the presence of certain risk factors that are well known in the art. Such risk factors include, without limitation, a genetic predisposition, a personal disease history, a familial disease history, a lifestyle factor, an environmental factor, a diagnostic indicator, and the like.

In some embodiments, the subject can be asymptomatic. Asymptomatic subjects include healthy subjects who have essentially no risk or only a low risk of developing RA (e.g., there is a less than 10%, less than 5%, less than 3%, or less than 1% probability that the asymptomatic patient will develop RA over the following five year period). Asymptomatic subjects further include healthy subjects who have a high risk of developing RA (e.g., there is a greater than 50%, greater than 70%, greater than 90%, or greater than 95% probability that the asymptomatic patient will develop RA over the following five year period). Asymptomatic subjects further include diseased subjects, who can display mild early diagnostic indicators of RA, but who are otherwise disease or complaint free (e.g., no synovial joint pain, no systemic inflammatory disorder). In some embodiments, the asymptomatic patient can be an arthralgia patient.

In some embodiments, the subject can have RA. In some embodiments, the subject can have RA with joint pain. In some embodiments, the subject can have RA with a systematic inflammatory disorder. In some embodiments, the subject can have juvenile idiopathic arthritis (JIA). In some embodiments, the subject can have a pre-RA syndrome. In some embodiments, the pre-RA syndrome can be arthralgia.

In some embodiments, the subject can be suspected of having RA. As used herein, a subject can be “suspected of having RA” as determined by the presence of certain risk factors that are well known in the art. Such risk factors include, without limitation, a genetic predisposition, a personal disease history, a lifestyle factor, an environmental factor, a diagnostic indicator and the like.

In some embodiments, the subject can be at risk of developing RA. In some embodiments, the subject can have a genetic predisposition for developing RA or a family history of RA or other autoimmune diseases. In some embodiments, the subject can be exposed to certain lifestyle factors (e.g., smoking cigarettes) promoting the development of RA or the subject can show clinical disease manifestations of RA. In some embodiments, the subject can be a patient who is receiving a clinical workup to diagnose RA or to assess the risk of developing RA.

In some embodiments, the subjects can have anti-PAD, that includes, for example, anti-PAD1 or anti-PAD1 and anti-PAD4, present, e.g., in their blood or another bodily tissue or fluid, (anti-PAD positive subjects). In some embodiments, the subjects can have elevated anti-PAD levels, e.g., in their blood or another bodily tissue or fluid, relative to normal healthy subjects. In some embodiments, the subjects can have no anti-PAD present, e.g., in their blood or another bodily tissue or fluid (anti-PAD-negative subjects).

In some embodiments, the subjects can have anti-PAD1 present, e.g., in their blood or another tissue or bodily fluid, (anti-PAD1 positive subjects) or the subjects can have elevated anti-PAD1 levels, e.g., in their blood or another tissue or bodily fluid, relative to normal healthy subjects. In some embodiments, the subjects can be negative for anti-PAD1.

In some embodiments, the subjects can have anti-PAD1 and anti-PAD4 present, e.g., in their blood or another tissue or bodily fluid, (anti-PAD1 and anti-PAD4 positive subjects) or the subjects can have elevated anti-PAD1 and anti-PAD4 levels, e.g., in their blood or another tissue or bodily fluid, relative to normal healthy subjects. In some embodiments, the subjects can be negative for anti-PAD1 and anti-PAD4.

In some embodiments, the subject can be treatment naïve. In some embodiments, the subject can be undergoing treatments for RA (e.g., drug treatments). In some embodiments, the subject can be in remission. In some embodiments, the remission can be drug-induced. In some embodiments, the remission can be drug-free.

In some embodiments, the subject can be an animal model for RA. In some embodiments, the animal model can be a mouse, rabbit, or primate model of RA. In some embodiments, the animal model can involve inducing anti-PAD that includes, for example, anti-PAD1 or anti-PAD1 and anti-PAD4, responses by immunizing or vaccinating an animal with PAD.

It should be noted that the terms “healthy control individual,” “healthy subjects,” and grammatical equivalents herein are used interchangeably and refer to subjects who do not have increased anti-PAD levels, RA or joint erosion above baseline or a standard known or determined to represent non-RA subjects.

The baseline or standard which determines or defines a subject as a non-RA subject is the reference interval. In diagnostic or health-related fields, the reference interval is a range of values observed in the reference subjects, which can be healthy control individuals, designated by specific percentiles. The reference interval can be any range of values as determined by those having skill in the art. See CLSI, “How to define and determine reference intervals in the clinical laboratory: approved guideline,” C28:A2 (2000). In some cases, the reference interval can be stringent or less stringent depending on the specific analyte being measured or disease being studied. A person having skill in the art will understand the appropriate stringency to use when determining the reference interval. Thus, in some embodiments, the reference interval can be set at the 95th percentile. In order to increase specificity and decrease sensitivity, e.g. increase stringency, a higher cut-off can be used such as the 96th percentile or the 97th, or the 98^{th}, or the 99^{th}.

In the present disclosure, anti-PAD, such as for example anti-PAD1 or anti-PAD1 and anti-PAD4, can be considered increased in a subject if the specific type of anti-PAD levels are at least above the 95th percentile relative to the corresponding specific type of anti-PAD levels in healthy control subjects. In other embodiments, anti-PAD, such as for example anti-PAD1 or anti-PAD1 and anti-PAD4 can be considered increased in a subject if the specific type of anti-PAD levels are above the 96^{th}, 97^{th}, 98^{th} or 99^{th} percentile.

In some embodiments, the presence of anti-PAD, such as for example anti-PAD1 or anti-PAD1 and anti-PAD4, can be based on a comparison of signal against background in a healthy subject. In some embodiments, the presence of anti-PAD, can be increased or decreased relative to an average or median anti-PAD level observed in a population of healthy subjects. In some embodiments, anti-PAD, such as for example anti-PAD1 or anti-PAD1 and anti-PAD4, can be absent in healthy subjects. In some embodiments, anti-PAD level cannot be detected above the noise of the respective assay used to determine anti-PAD level. In some embodiments, anti-PAD can be considered present in a sample if an anti-PAD level can be detected above the noise of the respective assay used to determine an anti-PAD level. In some embodiments, anti-PAD can be considered increased in a sample if the signal in an anti-PAD detection assay is at least two standard deviations above noise such as the average or mean signal for control samples. In some embodiments, anti-PAD can be considered present in a sample if the level of anti-PAD exceeds a predetermined threshold level. An anti-PAD threshold level can be determined by a skilled artisan, such as a clinical physician, based on a variety of factors, such as the specific objectives of a clinical trial or the diagnostic and prognostic significance of a certain anti-PAD level or the results of another diagnostic test for RA that does not involve the detection of anti-PAD levels.

In some embodiments, the present disclosure provides a polypeptide including a PAD or antigenic fragment thereof. The PAD can be used in the methods provided herein or included in the kits provided herein.

A PAD or antigenic fragment thereof can be obtained using various methods well known in the art. For example, a PAD or antigenic fragment thereof can be isolated from a natural source, produced by chemical synthesis or produced by recombinant protein expression.

In some embodiments, the PAD protein or antigenic fragment thereof is obtained by a method comprising isolation from a natural source, chemical synthesis or recombinant expression. In certain embodiments, the PAD protein or antigenic fragment thereof is obtained by a method comprising isolation from a natural source. In certain embodiments, the PAD protein or antigenic fragment thereof is obtained by a method comprising recombinant expression. In specific embodiments, the PAD protein or antigenic fragment thereof is obtained by chemical synthesis. In specific embodiments, the PAD protein is obtained by a method described in the Examples, infra.

In some embodiments, the PAD protein or antigenic fragment thereof is obtained by a method comprising recombinant expression, and calcium is used. In some embodiments, the PAD protein or antigenic fragment thereof is obtained by a method comprising recombinant expression, and calcium is not used.

In some embodiments, the PAD protein or antigenic fragment thereof is a citrullinated PAD protein or antigenic fragment thereof. In other embodiments, the PAD protein or antigenic fragment thereof is a citrullinated PAD protein or antigenic fragment thereof.

Exemplary methods for expressing and purifying recombinant polypeptides, for purifying polypeptides from cells, tissues or bodily fluids, and for chemically synthesizing polypeptides are well known in the art and can be found described in Scopes R. K., Protein Purification—Principles and Practice, Springer Advanced Texts in Chemistry, 3rd Edition (1994); Simpson R. J. et al., Basic Methods in Protein Purification and Analysis: A Laboratory Manual, Cold Spring Harbor Laboratory Press, 1st Edition (2008); Green M. R. and Sambrook J., Molecular Cloning: A Laboratory Manual, Cold Spring Harbor Laboratory Press, 4th Edition (2012); Jensen K. J. et al., Peptide Synthesis and Applications (Methods in Molecular Biology), Humana Press, 2nd Edition (2013).

Polypeptides purified or isolated from a natural source refers to the isolation and purification of a polypeptide from a source where it is naturally expressed. In some embodiments, a natural source of a PAD can be from a cell, tissue or bodily fluid of an organism. In some embodiments, the cells, tissues or bodily fluids can include, for example, whole blood, serum, plasma, synovial fluid or sputum from an organism of the present disclosure. A PAD or antigenic fragment thereof can similarly be isolated from any biological sample described and provided herein.

It should be noted that the terms “purified” or “isolated” refer to a polypeptide that is isolated, partially or completely, from a complex mixture of components, as found in nature. Thus, in some embodiments, a PAD of the present disclosure can be partially purified or substantially purified. Partial purification results in isolation from one or more components as found in nature. Substantial purification results in isolation from all components as found in nature. Partial purification, as disclosed herein, can be achieved by the methods and compositions provided herein. In some embodiments, a partially purified PAD can be performed with a capture probe. In some embodiments, the capture probe is a polypeptide or functional fragment thereof specific to PAD. In some embodiments, the capture probe is an anti-PAD antibody. Substantial purification, as exemplified herein, can be achieved by methods known in the art. In some embodiments, a PAD is purified substantially by a process of extraction, precipitation and solubilization.

Recombinant polypeptides can be expressed in and purified from bacterial cells (e.g., E. coli), yeast cells (e.g., S. cerevisiae), insect cells (e.g., Sf9), in mammalian cells (e.g., CHO) and others. Recombinant polypeptides can be expressed and purified as fusion proteins including tags for protein detection or affinity purification tags (e.g., His-tag, GST-tag, Myc-tag), including cleavable tags (e.g., tags including a TEV-cleavage site). In some embodiments, the PAD provided herein can be purified from a cell, tissue or bodily fluid obtained from an organism. Tissues or bodily fluids can include any tissue or bodily fluids obtained from the organism. In some embodiments, the tissues or bodily fluids can include blood, serum, plasma, synovial fluid, urine or milk (e.g., from goats, cows, sheep). One skilled in the art will recognize that methods for the purification of polypeptides from cells, tissues or bodily fluids are well known in the art.

In some embodiments, a PAD or antigenic fragment thereof is chemically synthesized using, for example, methods described in Jensen, K. J. (supra).

In some embodiments, a PAD antigenic fragment can be produced by enzymatically digesting full-length PAD. The full-length PAD can be obtained by, for example, any of the exemplary methods described above. The enzymatic digest can be carried out with, for example, a protease or peptidase. In some embodiments, the protease or peptidase can be an exoprotease or an exopeptidase. In some embodiments, the protease or peptidase can be an endoprotease or endopeptidase. In some embodiments, the protease or peptidase can include a serine protease, threonine protease, cysteine protease, aspartate protease, glutamic acid protease, or metalloprotease. In some embodiments, the protease or peptidase can include trypsin, chymotrypsin, pepsin, papain and any cathepsin including cathepsin B, L, D, K, or G.

In some embodiments, a PAD or antigenic fragment thereof can be a native PAD. In some embodiments, the PAD or antigenic fragment thereof can be a denatured or unfolded PAD. In some embodiments, the PAD or antigenic fragment thereof can include unnatural amino acids. In some embodiments, the unnatural amino acids can be methylated at the α-amino-group to produce polypeptides with methylated backbones. In some embodiments, the unnatural amino acids can be R-amino acids. In some embodiments, the unnatural amino acids can include dyes (e.g., fluorescent dyes) or affinity tags. In some embodiments, the PAD or antigenic fragment thereof can include chemical modifications. Chemical modifications can include, e.g., chemical modifications with biotin, fluorescent dyes. A skilled artisan will recognize that methods for introducing unnatural amino acids into polypeptides and for chemically modifying polypeptides are well known in the art.

In some embodiments, an isolated, chemically synthesized or recombinant PAD or antigenic fragment thereof can be a plurality of PADs. It should be noted that the term “plurality” refers to a population of two or more members, such as polypeptide members or other referenced molecules. In some embodiments, the two or more members of a plurality of members can be the same members. For example, a plurality of polypeptides can include two or more polypeptide members having the same amino acid sequence. By way of exemplification, a plurality of members having the same amino acid sequence can include two or more members of any one of PAD exemplified in Table 1. In some embodiments, the two or more members of a plurality of members can be different members. For example, a plurality of polypeptides can include two or more polypeptide members having different amino acid sequences. By way of exemplification, a plurality of members having different amino acid sequences can include at least one member of two or more PADs exemplified in Table 1. A plurality includes 2, 3, 4, 5, 6, 7, 8, 9, 10, 20, 30, 40, 50, 60, 70, 80, 90 or a 100 or more different members. A plurality can also include 200, 300, 400, 500, 1000, 5000, 10000, 50000, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶ or 1×10⁷ or more different members. A plurality includes, for example, all integer numbers in between the above exemplary plurality numbers. In some embodiments, a PAD can be a plurality of PADs from the organisms of the present disclosure.

As provided herein, RA can be determined in subjects of the present disclosure by the detection of anti-PAD1, or anti-PAD1 and anti-PAD4. In some embodiments, detection can further include anti-PAD3, anti-PAD2, and/or anti-PAD6. Detection of any of the anti-PAD described herein can be performed through the use of, for example, an antibody specific to IgG. An IgG binding molecule in the art can be used. In addition, an antibody specific to IgA can also be used for the detection of anti-PAD1 or anti-PAD1 and anti-PAD4. In some embodiments, a combination of IgG and IgA detection may be performed.

A label of the present disclosure can be conjugated to any of the detection probes identified herein. Conjugation can include non-covalent or covalent cross-linkage as described above. In some configurations, a label conjugated to a detection probe requires an additional substrate or binding agent described above. As an example, an HRP label conjugated to a detection probe requires a substrate, disclosed above, to detect a detection probe. Numerous other configurations for a label are known in the art. The present disclosure includes all label configurations exemplified herein and/or known in the art. In some embodiments, a label configuration can include PE conjugated to a PAD (e.g., PAD1 or PAD1 and PAD4), a PAD:anti-PAD complex binding agent, an anti-PAD IgG, an anti-IgG, an anti-PAD IgA or an anti-IgA. In alternative embodiments, a label configuration can include PE conjugated to a specific PAD including, for example, PAD1, or PAD1 and PAD4. In further embodiments, a label configuration can include a PE conjugated to an anti-PAD IgG including, for example, anti-PAD1 IgG, anti-PAD1 IgG and anti-PAD4 IgG, anti-PAD1 IgA, anti-PAD1 IgA and anti-PAD4 IgA, or combinations thereof.

Methods for detecting, measuring and/or quantifying a signal produced by a label of the present disclosure are well known in the art and include detection of fluorescence, luminescence, chemiluminescence or absorbance, reflectance, transmittance, birefringence or refractive index. Optical methods include imaging methods such as confocal and non-confocal microscopy and non-imaging methods such as microplate readers. In some embodiments, methods of detecting anti-PAD, such as, for example, anti-PAD1 or anti-PAD1 and anti-PAD4, in biological sample can include visualization, quantification or both of a fluorescent, colorimetric or absorbance signal in a biological sample.

In some embodiments of the present disclosure, anti-PAD, such as, for example, anti-PAD1 or anti-PAD1 and anti-PAD4, presence can be detected by immunoassay. Methods and protocols for conducting immunoassays and biophysical protein-interaction assays are well known in the art. See, e.g., Wild D., The Immunoassay Handbook, Elsevier Science, 4^{th} Edition (2013); Fu H., Protein-Protein Interactions, Humana Press, 4^{th} Edition (2004). Exemplary immunoassays include fluorescent immunosorbent assay (FIA), a chemiluminescent immunoassay (CIA), a radioimmunoassay (RIA), multiplex immunoassay, a protein/peptide array immunoassay, a solid phase radioimmunoassay (SPRIA), an indirect immunofluorescence assay (IIF), an enzyme linked immunosorbent assay (ELISA) and a particle based multianalyte test (PMAT), or a Dot Blot assay.

In some embodiments, the ELISA can be a sandwich ELISA. In some embodiments, the sandwich ELISA can include the initial step of immobilizing a purified polypeptide of this disclosure on a solid support as exemplified below. For example, a PAD or antigenic fragment thereof can be immobilized on a wall of a microtiter plate well or of a cuvette. In some embodiments, contacting the sample from the subject with the PAD or antigenic fragment thereof of this disclosure can include exposing the sample to the immobilized PAD or antigenic fragment thereof.

In some embodiments, the ELISA can be a direct ELISA. In some embodiments, the direct ELISA can include the initial step of immobilizing a PAD or antigenic fragment thereof on any of the solid supports disclosed herein. For example, a PAD or antigenic fragment thereof can be immobilized to a wall of a microtiter plate well or of a cuvette. In some embodiments, contacting the sample from the subject with a PAD or antigenic fragment thereof of this disclosure can include exposing the anti-PAD contained in the patient's sample to the immobilized PAD. Any of the immunoassays disclosed herein (see above) and in the art can be used, or modified to be used, in any of the methods disclosed herein.

In some embodiments, anti-PAD, such as anti-PAD1 or anti-PAD1 and anti-PAD4, can be detected by a particle based multianalyte test. For example, in PMAT, different types of particles are used simultaneously, with each type having immobilized a specific binding partner for a specific molecule species on the surface of its particles. In a solution, the analyte molecules to be detected are bound to their binding partners on the corresponding particle type. The bonds are then detected optically through the addition of a secondary marker that marks all particle-bound analyte molecules of the multiplex assay. A PMAT can be performed using a variety of formats known in the art, such as flow cytometry, a capture sandwich immunoassay, or a competitive immunoassay. For example, using a dual-laser flow-based detection instrument, the binding of analyte fractions, such as autoantibodies, can be detected through the fluorescence of the secondary marker. In some embodiments, the PMAT particle can be a bead. In effecting the PMAT, the presence of one or more autoantibodies specifically associated with an autoimmune disease can be identified, and the patient can be diagnosed with the autoimmune disease that is specifically associated with the autoantibody identified by the PMAT.

In some embodiments, a Dot-Blot or line immunoassay (LIA) can be used to detect anti-PAD, such as anti-PAD1 or anti-PAD1 and anti-PAD4, in a biological sample. Methods and protocols for dot blot are well known in the art, including estimating polypeptide concentration. See Joint ProteomicS Laboratory (JPSL) of the Ludwig Institute for Cancer Research, Estimating protein concentration by dot blotting of multiple samples, Cold Spring Harbor Protocols, New York (2006).

In some embodiments, the immunoassay can be performed by immobilizing a capture probe to a solid support for a sufficient time to allow binding to occur. A capture probe includes a binding agent that binds to an analyte of interest. With respect to detection of an anti-PAD, such as, for example, anti-PAD1 or anti-PAD1 and anti-PAD4, a capture probe can be any binding agent that specifically binds to anti-PAD, PAD:anti-PAD complex or anti-PAD. Exemplary capture probes includes, PAD and/or a particular PAD such as PAD1, PAD2, PAD3 and/or PAD4, as well as antigenic fragments thereof. Other exemplary capture probes include anti-IgG antibodies and/or anti-IgA antibodies and functional fragments thereof, anti-IgG and/or IgA binding polypeptides and functional fragments thereof, anti-PAD IgG and/or anti-PAD IgA binding polypeptides, including antibodies, and functional fragments thereof and/or PAD:anti-PAD complex binding polypeptides and functional fragments and binding agents.

The immunoassay can further include blocking steps, washing steps and additionally or alternatively, elution steps. Blocking steps can include contacting a solid support of the immunoassay in a blocking buffer for a sufficient time and temperature to allow blocking. Exemplary blocking buffers are identified below as are exemplary solid supports. Washing steps include contacting a solid support of the immunoassay with a washing buffer to remove non-specific binding of polypeptides to the solid support. Exemplary washing buffers are described below. Elution buffers can include any of a variety of elution buffers known in the art or disclosed herein. Elution buffers include, for example, a 0.1 M glycine:HCl solution between pH 2.5 and 3. Polypeptide complexes can be eluted from the solid support of the immunoassay to aid in detection and measurement of, for example, PAD and anti-IgG complexes or PAD and anti-IgA.

The present disclosure also provides a kit which can be used to diagnosis RA, or monitor RA progression. The kit can include at least one peptidyl arginine deiminase (PAD) protein, or an antigenic fragment thereof, that can capture an autoantibody specific to the PAD protein; a detection probe that recognizes said autoantibody, and a solid support, and the at least one PAD protein can include PAD1 or PAD1 and PAD4. In specific embodiments, the at least one PAD protein is PAD1 or an antigenic fragment thereof. In other embodiments, the at least one PAD protein is PAD1 and PAD4 or an antigenic fragment thereof.

In some embodiments, the kit of the present disclosure that includes PAD1 or PAD1 and PAD4 can further include one or more PAD protein selected from the group consisting of PAD2, PAD3, and PAD6 or an antigenic fragment thereof. In specific embodiments, the at least one PAD protein is PAD1, PAD4, and PAD2 or an antigenic fragment thereof. In other embodiments, the at least one PAD protein is PAD1, PAD4, and PAD3 or an antigenic fragment thereof. In still other embodiments, the at least one PAD protein is PAD1, PAD4, PAD2, and PAD3 or an antigenic fragment thereof. In further embodiments, the at least one PAD protein is PAD1, PAD4, PAD2, PAD3, and PAD6 or an antigenic fragment thereof.

The kit can include any of the detection probes provided herein as well as others well known in the art. For example, a detection probe can include an antibody or a ligand. A detection probe can be immobilized on a solid support. It should be noted that the term “immobilized” is used interchangeably with “attached” and both terms are intended to include covalent and non-covalent attachment, unless indicated otherwise, either explicitly or by context. In some embodiments, a PAD protein or antigenic fragment thereof is immobilized to a solid support.

As exemplified with respect to the methods of this disclosure, a kit can include any of the labels described or exemplified herein. For example, a label of the kit can include a fluorophore, an enzyme, a chemiluminescent moiety, a radioactive moiety, an organic dye, a small molecule, a polypeptide or functional fragment thereof. In some embodiments, a label of the kit includes PE. In some embodiments, a label of the kit includes FITC. In some embodiments, a label of the present disclosure is conjugated to a detection probe of the disclosure as exemplified above.

A kit can include any solid support provided herein or identified in the art. As used herein, the terms “solid support,” “solid surface” and other grammatical equivalents refer to any material that is appropriate for or can be modified to be appropriate for the attachment of PAD, such as PAD1 or PAD1 and PAD4, or an antigenic fragment thereof of this disclosure. Possible materials include, without limitation, glass and modified or functionalized glass, plastics (including acrylics, polystyrene, methylstyrene, polyurethanes, Teflon™, etc.), paramagnetic materials, thoria sol, carbon graphite, titanium oxide, latex or cross-linked dextrans such as Sepharose, cellulose polysaccharides, nylon or nitrocellulose, ceramics, resins, silica or silica-based materials including silicon and modified silicon, carbon metals, inorganic glasses, optical fiber bundles, and a variety of other polymers. In some embodiments, the solid supports can be located in microtiter well plates (e.g., a 96-well, 384-well or 1536-well plate). In some embodiments, the solid supports can be located within a flow cell or flow cell apparatus (e.g., a flow cell on a Biacore™ chip or a protein chip).

In some embodiments, the solid support can be a bead, microsphere, particle, membrane, chip, slide, well, and test tube. Beads include microspheres or particles. By “microspheres” or “particles” or grammatical equivalents herein is meant small, discrete, non-planar particles in the micrometer or nanometer dimensions. In some embodiments the bead can be spherical, in other embodiments the bead is irregular. Alternatively or additionally, the beads can be porous. The bead sizes range from nanometers to millimeters with beads from about 0.2 to about 200 microns being preferred in some embodiments. In other embodiments, bead size can range from about 0.5 to about 5 microns. In some embodiments, beads smaller than 0.2 microns and larger than 200 microns can be used. In some embodiments, the solid support can include an array of wells or depressions in a surface. This can be fabricated as is known in the art using a variety of techniques, including, photolithography, stamping techniques, molding techniques and microetching techniques. As will be appreciated by those skilled in the art, the technique used will depend on the composition and shape of the array substrate.

In some embodiments, the solid support can include a patterned surface suitable for immobilization of purified proteins in an ordered pattern (e.g., a protein chip). A “patterned surface” refers to an arrangement of different regions in or on an exposed layer of a solid support. For example, one or more of the regions can be features where one or more purified proteins are present. The features can be separated by interstitial regions where purified proteins are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. App. Publ. No. 2008/0280785 A1, U.S. Pat. App. Publ. No. 2004/0253640 A1, U.S. Pat. App. Publ. No. 2003/0153013 A1 and International Publication No. WO 2009/039170 A2.

In some embodiments, a solid support can have attached to its surface a PAD, such as, for example, PAD1 or PAD1 and PAD4, or an antigenic fragment thereof. In some embodiments, any PAD exemplified by, for example, Tables 1-3, including antigenic fragments thereof can be attached to a solid support. In some embodiments, any PAD or antigenic fragment thereof of the present disclosure can be immobilized to a solid support via a linker molecule. In some embodiments, all that is required is that molecules, such as any PAD or antigenic fragment thereof of the present disclosure, remain immobilized or attached to the support under the conditions in which it is intended to use the support, for example, in applications requiring antibody binding or detection.

A kit can include a positive control. In some embodiments, a positive control can be a sample containing a detectable amount of anti-PAD, such as, for example, anti-PAD1 or anti-PAD1 and anti-PAD4, or levels above the threshold. In some embodiments, a positive control can be obtained from a diseased subject who has levels of anti-PAD above threshold. Additionally or alternatively, a positive control can contain anti-PAD synthesized in vitro using any of the methods described herein. In other embodiments, the kit can include a negative control. A negative control can be a sample containing no detectable amount of anti-PAD or levels below the threshold. In some embodiments, a negative control can be obtained from a healthy control individual or can be synthesized in vitro. For example, a negative control can include water or buffer.

The kit or the disclosure can further include one or more ancillary reagents. As used herein, “ancillary reagents” refer to a substance, mixture, material or component that is useful to carry out an intended purpose of the kit. Ancillary reagents can include a reagent, including a conjugation reagent, a buffer, standard, positive control, label, instructions and the like.

As provided herein and exemplified with respect to the methods of this disclosure, a kit of this disclosure can include a reporter tag. Reporter tags function to produce a signal for detection of a biomarker. Reporter tags can be attached, for example, to any of the detection probes used herein through non-covalent or covalent cross-linkage.

In some embodiments, a reagent of the kit of the present disclosure can include any conjugation reagent known in the art, including covalent and non-covalent conjugation reagents. Covalent conjugation reagents can include any chemical or biological reagent that can be used to covalently immobilize a polypeptide of this disclosure on a surface. Covalent conjugation reagents can include a carboxyl-to-amine reactive group such as carbodiimides such as EDC or DCC, an amine reactive group such as N-hydroxysuccinimide (NHS) ester or imidoesters, a sulfhydryl-reactive crosslinker such as maleimides, haloacetyls, or pyridyl disulfides, a carbonyl-reactive crosslinker groups such as, hydrazides or alkoxyamines, a photoreactive crosslinker such as aryl azides or dizirines, or a chemoselective ligation group such as a Staudinger reaction pair. Non-covalent immobilization reagents can include any chemical or biological reagent that can be used to immobilize a polypeptide of this disclosure non-covalently on a surface, such as affinity tags such as biotin or capture reagents such as streptavidin or anti-tag antibodies, such as anti-His6 or anti-Myc antibodies.

The kits of this disclosure can include combinations of conjugation reagents. Such combinations include, e.g., EDC and NHS, which can be used, e.g., to immobilize a protein of this disclosure on a surface, such as a carboxylated dextrane matrix (e.g., on a BIAcore™ CM5 chip or a dextrane-based bead). Combinations of conjugation reagents can be stored as premixed reagent combinations or with one or more conjugation reagents of the combination being stored separately from other conjugation reagents.

In other embodiments, a reagent of the kit can include a reagent such as a coating buffer. A coating buffer can include sodium carbonate-sodium hydroxide or phosphate. In some embodiments, the coating buffer can be 0.1M NaHCO₃ (e.g., about pH 9.6).

In some embodiments, a reagent of a kit can include a washing buffer. A washing buffer can include tris (hydroxymethyl)aminomethane (Tris)-based buffers like Tris-buffered saline (TBS) or phosphate buffers like phosphate-buffered saline (PBS). Washing buffers can be composed of detergents, such as ionic or non-ionic detergents. In some embodiments, the washing buffer can be a PBS buffer at about pH 7.4 including Tween®20 at about 0.05%. In other embodiments, the washing buffer can be the BIO-FLASH™ Special Wash Solution (Inova Diagnostics, Inc., San Diego, Calif.).

In some embodiments, a reagent of the kit can include a dilution buffer. Any dilution buffer known in the art can be included in the kit of the present disclosure. Typical dilution buffers include a carrier protein such as bovine serum albumin (BSA) and a detergent such as Tween®20. In some embodiments, the dilution buffer can be PBS at about pH 7.4 including BSA at about 1% BSA and Tween®20 at about 0.05%.

In some embodiments, a reagent can include a detection or assay buffer. Any detection or assay buffer known in the art can be included in the kit of the present disclosure. The detection or assay buffer can be a colorimetric detection or assay buffer, a fluorescent detection or assay buffer or a chemiluminescent detection or assay buffer. Colorimetric detection or assay buffers include PNPP (p-nitrophenyl phosphate), ABTS (2,2′-azino-bis(3-ethylbenzothiazoline-6-sulphonic acid)) or OPD (0-phenylenediamine). Fluorescent detection or assay buffers include QuantaBlu™ or QuantaRed™ (Thermo Scientific, Waltham, Mass.). Chemiluminescent detection or assay buffers can include luminol or luciferin. Detection or assay buffers can also include a trigger such as H₂O₂ and a tracer such as isoluminol-conjugate. In some embodiments, the detection reagent can include one or more BIO-FLASH™ Trigger solutions (Inova Diagnostics, Inc., San Diego, Calif.). In some embodiments, a reagent of the kit of the present disclosure can include solutions useful for calibration or testing.

In some embodiments, a reagent of the kit can include a stop solution. Any stop solution known in the art can be included in a kit of this disclosure. The stop solutions of this disclosure terminate or delay the further development of the detection reagent and corresponding assay signals. Stop solutions can include, e.g., low-pH buffers (e.g., glycine-buffer, pH 2.0), chaotrophic agents (e.g., guanidinium chloride, sodium-dodecylsulfate (SDS)) or reducing agents (e.g., dithiothreitol, β-mecaptoethanol), or the like.

In some embodiments, a reagent of the kit of this disclosure can include cleaning reagents. Cleaning reagents can include any cleaning reagent known in the art. In some embodiments, the cleaning reagents can be the cleaning reagents recommended by the manufacturers of the automated assay systems. In some embodiments, the cleaning reagents can include the BIO-FLASH™ System Rinse or the BIO-FLASH™ System Cleaning solutions (Inova Diagnostics, Inc., San Diego, Calif.).

A detection probe of the kit can include any of the detection probes described above. In brief, a detection probe of the kit can include antibodies and ligands. Thus, a detection probe specific for anti-PAD includes, for example, PAD, a PAD:anti-PAD complex binding agent, an anti-PAD IgG and/or anti-PAD IgA binding agent and an IgG and/or IgA binding agent. The anti-PAD IgG detection probes include binding agents to anti-PAD1 IgG, anti-PAD2 IgG, anti-PAD3 IgG, anti-PAD4 IgG, and/or anti-PAD6 IgG. The anti-PAD IgA detection probes include binding agents to anti-PAD1 IgA, anti-PAD2 IgA, anti-PAD3 IgA, anti-PAD4 IgA, and/or anti-PAD6 IgA.

A detection probe of the kit can be conjugated to any of the labels previously disclosed herein. For example, a detection probe can be conjugated to a fluorophore, an enzyme, a chemiluminscent moiety, a radioactive moiety, an organic dye, a small molecule, a polypeptide or functional fragment thereof. Examples of fluorophores include fluorescent dyes like phycoerytherin (PE), fluorescein isothiocyanate (FITC), tetramethylrhodamine (TRITC), BODIPY and AlexaFluor® dyes. Fluorescent dyes can also include fluorescence resonance energy transfer (FRET)-dyes or time-resolved (TR)-FRET dyes. Fluorophore labels also include fluorescent proteins such as green fluorescent protein (GFP) and cyan fluorescent protein (CFP). Examples of enzyme labels include alkaline phosphatase (AP) or horseradish peroxidase (HRP). When any of the substrates 3,3′5,5′-Tetramethylbenzidine (TMB), 3,3′-Diaminobenzidene (DAB), or 2,2′-azino-bis(3-ethylbenzothiazoline-6-sulphonic acid) (ABTS) are applied to HRP, a colored (chromogenic) or light (chemiluminescent) signal is produced. Radioactive moiety labels include carbon-14 or Tritium. Small molecule labels include biotin, resins such as agarose beads and fluorescently labeled magnetic beads, or nanoparticles such as colloidal gold. Polypeptide or functional fragment labels include Avidin, Streptavidin or NeutrAvidin which have an affinity for biotin. Polypeptide or functional fragment labels also include hemagglutinin (HA), glutathione-S-transferase (GST) or c-myc.

In some embodiments, the kit provided in this disclosure can include a component suitable for collecting a biological sample. A component can include collection tubes, columns, syringes, needles and the like. In some embodiments, the kit can include instructions for using the components of the kit. Instructions can be in any form, inside or outside of the kit. The instructions provide details regarding protocol and analytical techniques.

In some embodiments, a kit of the disclosure can include an instrument to an automated assay system. Automated assay systems can include systems by any manufacturer. In some embodiments, the automated assay systems can include, e.g., the BIO-FLASH™, the BEST 2000™, the DS2™, the ELx50 WASHER, the ELx800 WASHER, the ELx800 READER, and the Autoblot S20™ (Inova Diagnostics, Inc., San Diego, Calif.). In other embodiments, an instrument of the kit can be a detection instrument. A detection instrument can include any detection instrument in the art. Detection instruments are capable of detecting or measuring a label of the reporter tags of the present disclosure. Thus, detection instruments are capable of detecting or measuring fluorescence, luminescence, chemiluminescence or absorbance, reflectance, transmittance, birefringence or refractive index. In some embodiments, detection instruments can include confocal and non-confocal microscopy, a microplate reader, a flow cytometer and the like.

Components of a kit of the disclosure can be in varying physical states. For example, some or all of the components can be lyophilized or in aqueous solution or frozen. Such components include a PAD, such as PAD1 or PAD1 and PAD4, a detection probe, and ancillary reagents. Ancillary reagents include immobilization buffer, incubation buffer, washing buffer, dilution buffer, detection or assay buffer and blocking buffer. A person skilled in the art recognizes that there are various types of incubation, washing, detection and blocking buffers.

A kit of this disclosure can be tailored to specific assay technologies. In some embodiments, a kit can be tailored to assay technologies exemplified herein. For example, in some embodiments, the kits can be a FIA kit, a CIA kit, a RIA kit, a multiplex immunoassay kit, a protein/peptide array immunoassay kit, a SPRIA kit, an IIF kit, an ELISA, a PMAT kit, or a Dot Blot kit. In some embodiments, the ELSA kits can include a washing buffer, a sample diluents, a secondary antibody-enzyme conjugate, a detection reagent and a stop solution. In some embodiments, the Dot Blot kits can include a washing buffer, a sample diluents, a secondary antibody-enzyme conjugate, a detection reagent, and a stop solution. In some embodiments, the CIA kit can include a washing buffer, a sample diluent, a tracer (e.g., isoluminol-conjugate) and a trigger (e.g., H₂O₂). In some embodiments, the multiplex kit can include a washing buffer, a sample diluent and a secondary antibody-enzyme conjugate. In some embodiments, the kits can be tailored to the Luminex platform and include, as an example, xMAP® beads.

A kit can be used to diagnose RA, or monitor RA, by providing a means for detecting anti-PAD, such as anti-PAD1 or anti-PAD1 and anti-PAD4, that is reactive with PAD, such as PAD1 or PAD1 and PAD4, respectively, or an antigenic fragment thereof. A kit can detect anti-PAD autoantibodies by any of the techniques described herein, as well as those known in the art. Complexes of anti-PAD and a PAD, or antigenic fragment thereof, can have a stoichiometry of one to one or more than one to one anti-PAD. In some embodiments, the complexes can have one anti-PAD antibody per PAD or antigenic fragment thereof. In some embodiments, the complexes can have two anti-PAD per PAD or antigenic fragment thereof. In some embodiments, the complexes can have more than two anti-PAD per PAD or antigenic fragment thereof. Methods for measuring binding stoichiometries of two antigens are well known in the art and include, e.g., isothermal titration calorimetry (ITC) and ultracentrifugation.

In some embodiments, the complexes of anti-PAD and PAD, or antigenic fragment thereof, can be a plurality of complexes with identical stoichiometry. For example, all complexes in the plurality of complexes have one anti-PAD per purified PAD or antigenic fragment thereof. In some embodiments, the complexes of anti-PAD and PAD or antigenic fragment thereof, can be a plurality of complexes with different stoichiometries. For example, some complexes in the plurality of complexes can have one anti-PAD per PAD or antigenic fragment thereof and some other complexes in the plurality of complexes can have more than one anti-PAD per PAD or antigenic fragment thereof.

In some embodiments, a PAD or antigenic fragment thereof can be bound by anti-PAD with higher affinity. In some embodiments, anti-PAD binding sites can be bound by anti-PAD with more than 2-fold, more than 3-fold, more than 4-fold, more than 5-fold, more than 8-fold, more than 10-fold, more than 15-fold, more than 20-fold, more than 25-fold, more than 50-fold, more than 100-fold, more than 300-fold, more than 1,000-fold, more than 3,000-fold, more than 10,000-fold, more than 30,000-fold, or more than 100,000-fold greater binding affinity. Greater binding affinities are evidenced by lower dissociation constants (K_{D}S) for anti-PAD-PAD complex or by higher association constants (K_{A}S) for the respective anti-PAD and PAD. In some embodiments, the dissociation constants for (K_{D}S) for the anti-PAD-PAD complexes can be less than 1 mM, less than 300 nM, less than 100 nM, less than 30 nM, less than 10 nM, less than 3 nM, less than 1 nM, less than 300 pM, less than 100 pM, less than 30 pM, less than 10 pM, less than 3 pM, or less than 1 pM. Methods for measuring binding affinities of antibodies to antigens are well known in the art and include ELISA, isothermal titration calorimetry (ITC) and surface plasmon resonance (SPR).

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

### EXAMPLES

### Example I

### Identification of PAD1 and PAD6 as Novel Antigenic Targets in Rheumatoid Arthritis (RA)

This example illustrates that PAD1 and PAD6 are novel antigenic target in RA, and that anti-PAD1 and anti-PAD6 can be used to identify RA patients.

To determine whether the presence of antibodies to any of the five known protein-arginine deiminase (PAD) family members (PAD1, PAD2, PAD3, PAD4, and PAD6) were able to discriminate between RA patients and non-RA controls, a panel was developed for the detection of anti-PAD IgG based on a particle-based multi-analyte technology (PMAT). The panel utilized paramagnetic particles coupled with the different human recombinant PAD proteins (PAD1, PAD2, PAD3, PAD4, and PAD6) and anti-human IgG conjugate. This panel was used to test a first cohort of patients (“Cohort I”) using sera from RA patients (n=33) and non-RA controls (n=36). The controls were comprised of apparently healthy individuals (n=10), and patients with infectious diseases (n=10), systemic lupus erythematosus (n=7), systemic sclerosis (n=9) and Sjögren's syndrome (n=1).

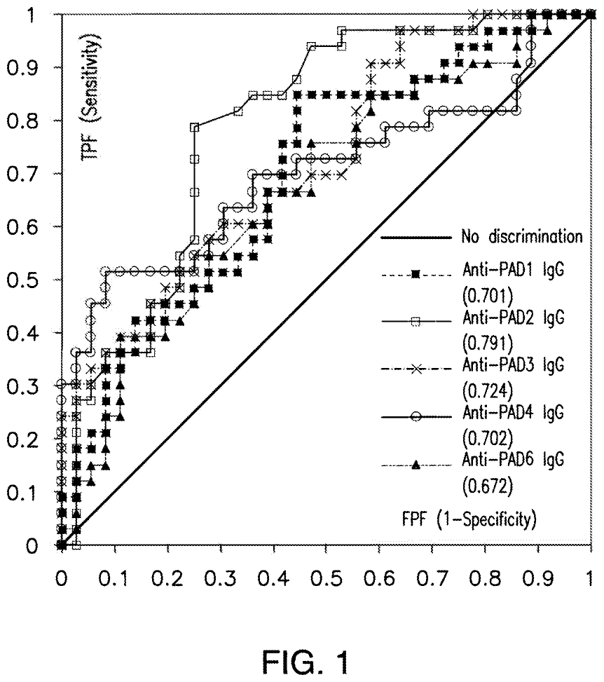

The results revealed that all five anti-PAD IgG demonstrated the ability to discriminate between RA patients and non-RA controls (FIG. 1). At greater than 90% specificity, anti-PAD4 IgG, followed by anti-PAD3 IgG, showed the best diagnostic performance. Significantly higher levels of the antibodies were observed in RA vs. non-RA controls for anti-PAD2, anti-PAD3, and anti-PAD4 (p-values of <0.0001, 0.0014, and 0.0039, respectively), which confirmed PAD2, PAD3, and PAD4 as autoantigens. Surprisingly, higher levels of anti-PAD1 and anti-PAD6 were also observed in RA vs non-RA controls (p-values of 0.0041, and 0.0140, respectively).

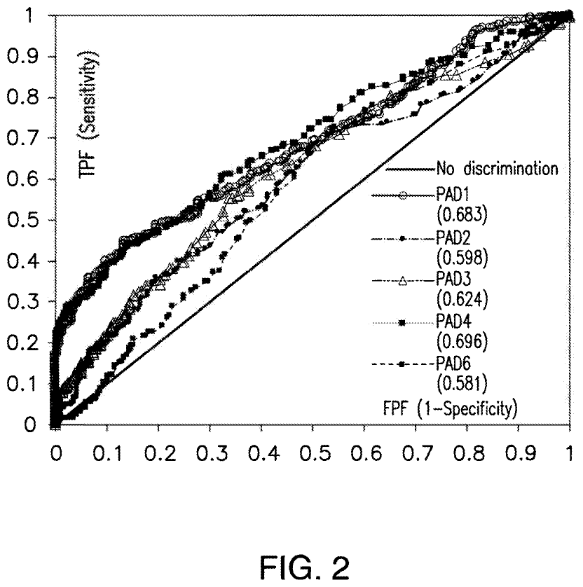

Similar results were also achieved in a larger study (“Cohort II”) that involved a total of 275 RA patients and 285 controls (FIG. 2). Notably, the discrimination between RA and non-RA controls was comparable for anti-PAD1 and anti-PAD4, even in the larger study.

Collectively, these results confirmed that identification of anti-PAD2, anti-PAD3, and anti-PAD4 were useful in discriminating RA patients from non-RA controls, and identified for the first time that PAD1 and PAD6 could also be useful for the same purpose.

### Example II

### Performance of Anti-PAD1 and Anti-PAD4 Detection Correlated in Sera of RA Patients

This example demonstrates that the performance of anti-PAD1 and anti-PAD4 strongly correlate in the sera of RA patients.

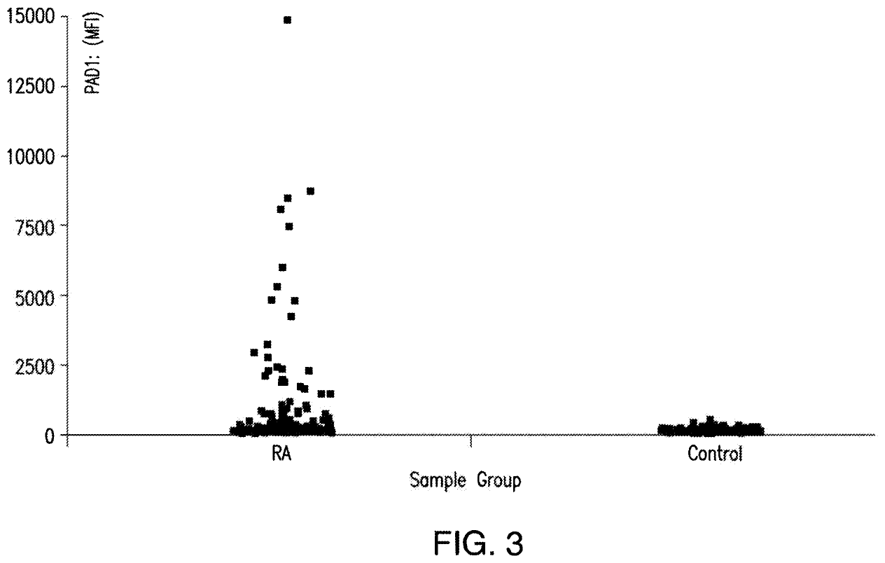

Analysis of the samples from Cohort I, described in Example I, revealed that while principal component analysis (PCA) showed an association between all anti-PAD antibodies, there was further discrimination that displayed closer association between anti-PAD1, 3 and 4 on one hand, and between anti-PAD2 and 6 (FIG. 3). Specifically, the highest correlation was between anti-PAD1 and anti-PAD4 (Spearman's rho=0.87, p<0.0001), and the lowest correlation was between anti-PAD4 and anti-PAD2 (Spearman's rho=0.38, p=0.0015), as well as between anti-PAD4 and anti-PAD6 (Spearman's rho=0.38, p=0.0011). These results were consistent with the similar performance of anti-PAD1 and anti-PAD4 observed in both Cohorts of patients (FIG. 1 and FIG. 2).

Collectively, the correlation results revealed that the anti-PAD1 strongly correlated with anti-PAD4, which is a known marker for RA.

### Example III

### Anti-PAD1 Detection Discriminated Against Disease Controls

This example demonstrates that detection of anti-PAD1 is also useful in identifying RA patients from among various diseases.

To determine whether anti-PAD1 was specific for RA, samples of sera from non-RA disease controls were compared to RA patients. The non-RA controls included samples from Hashimoto's disease (HD), idiopathic inflammatory myopathies (IIM), Sjögren's syndrome (SjS), ankylosing spondylitis (AS), healthy individuals (HI), juvenile idiopathic arthritis (JIA), psoriatic arthritis (PsA), systemic lupus erythematosus (SLE), chronic obstructive pulmonary disease (COPD), infectious diseases (ID), osteoarthritis (OA), and small vessel vasculitis (SVV). The results from testing against various non-RA controls, including different types of autoimmune diseases that commonly have autoantibodies, revealed that anti-PAD1 was specific for RA, with a sensitivity and specificity of approximately 30% and 97%, respectively (FIG. 3).

Thus, detection of anti-PAD1 was able to discriminate RA from other types of diseases, including different types of autoimmune disease, and represents a novel diagnostic marker for identifying patients having RA.

### Example IV

### Combining Detection of Anti-PAD1 with Detection of Additional Anti-PAD Autoantibodies Improved Diagnosis of RA

This example demonstrates that anti-PAD1 detection can be combined with detection of one or more additional anti-PAD autoantibodies to improve diagnosis of RA.

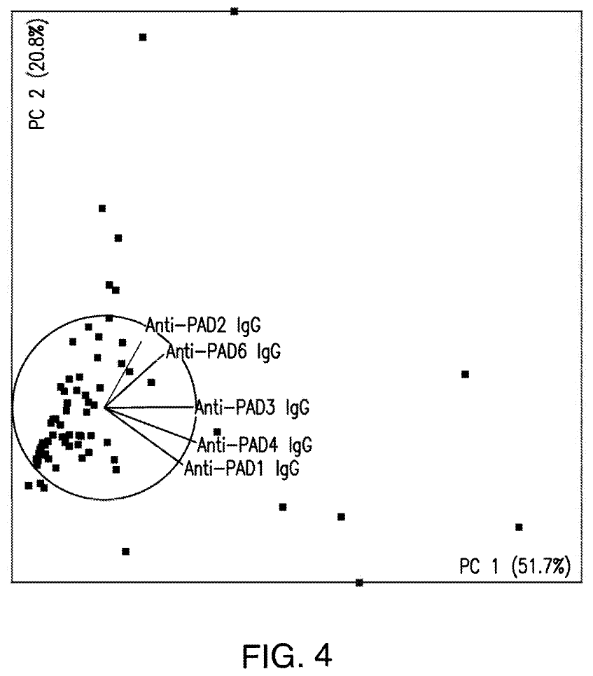

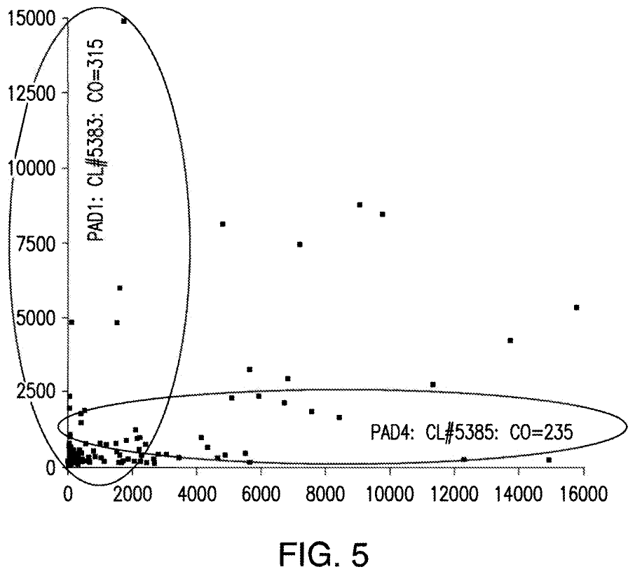

The performance of anti-PAD1 and anti-PAD4 detection in discriminating RA patients from non-RA patients was similar (FIG. 1-FIG. 2), and a strong correlation was observed among anti-PAD1 and anti-PAD4 (FIG. 4). Interestingly, despite the correlation between anti-PAD1 and anti-PAD4, analysis of the samples from Cohort II revealed that there are samples that either react with PAD1 or PAD4, with high levels (FIG. 5). This indicated that there may be exclusive epitopes between the antibodies rather than cross-reactivity between autoantibodies against PAD1 and PAD4, and that a novel method of combining detection of anti-PAD1 and anti-PAD4 together may improve the performance.

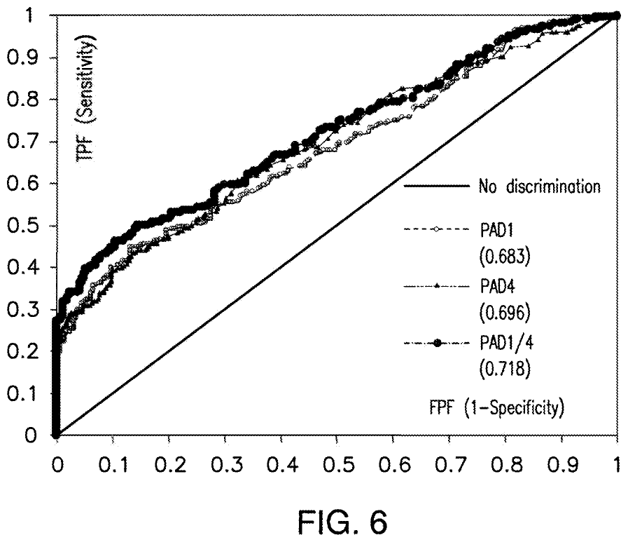

When the Cohort II samples were tested using PAD1 and PAD4, an improved performance over PAD1 or PAD4 alone was observed (FIG. 6). Specifically, detection of anti-PAD1 and anti-PAD4 had an area under the curve (AUC) of 0.718, whereas detection of anti-PAD1 alone had an AUC of 0.683 and detection of anti-PAD4 alone had an AUC of 0.696. Therefore, in addition to their usefulness as antibodies individually, the combination of PAD1 and PAD4 for the detection of anti-PAD1 and anti-PAD4, respectively, can improve the diagnosis of RA patients.

In addition, detection of anti-PAD1 antibodies was able discriminate between RA and non-RA patients combined with anti-PAD2 and anti-PAD6. The results indicated that detection of anti-PAD1, anti-PAD2 and anti-PAD6 was able to improve the diagnosis of RA compared to anti-PAD1 and anti-PAD4, alone or in combination (FIG. 6).

Taken together, these experiments revealed that combining the detection of anti-PAD1 and anti-PAD4 can improve the diagnosis of RA, as compared to either antibody alone. In addition, these results show that detection of anti-PAD1 can be combined with detection of other anti-PAD autoantibodies, and is not limited to being combined with anti-PAD4 for its usefulness in RA applications.

### Example V

### IgA and IgG Isotypes of Anti-PAD1 Identified in Sera of RA Patients

This example demonstrates that anti-PAD1 with IgG and IgA isotypes can be detected in RA patients, and that anti-PAD1 IgA is also able to discriminate RA from controls.

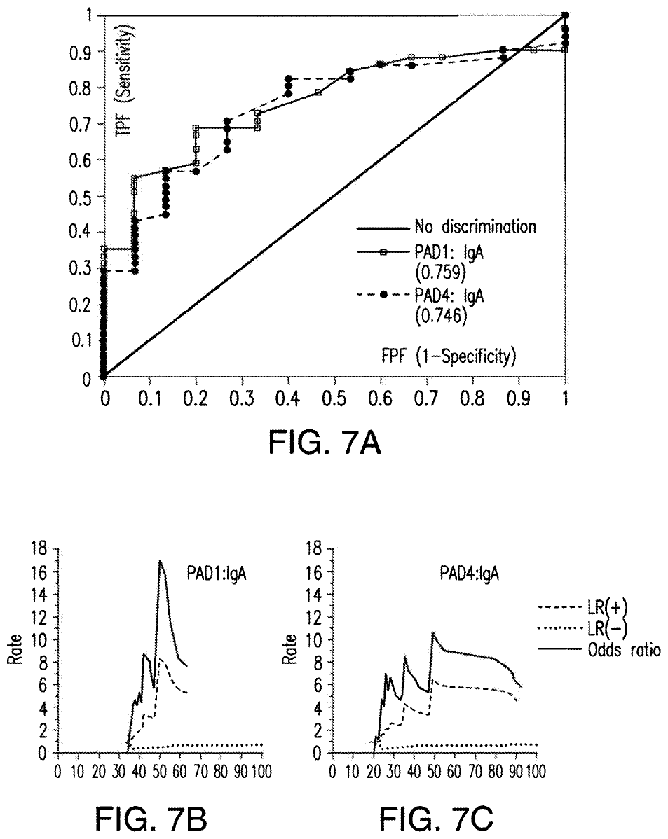

As shown above, anti-PAD1 IgG was found to be a useful biomarker in discriminating RA from non-RA controls, and that the combination of anti-PAD1 and anti-PAD4 was able to improve the diagnosis. To determine whether anti-PAD1 IgA was also able to discriminate RA from controls, a total of 51 RA patients and 15 controls were tested using PAD1 and PAD4 as antigens to assess the ability to discriminate RA from controls for anti-PAD1 IgA and anti-PAD4 IgA. The results revealed that PAD1 and PAD4 as antigens exhibited equal or superior performance for anti-PAD1 IgA vs anti-PAD4 IgA (FIG. 7A).

In addition, the likelihood and odds ratios (OR) were determined for both anti-PAD1 IgA and anti-PAD4 IgA. The results indicate significantly higher discrimination for anti-PAD1 IgA vs. anti-PAD4 IgA (FIG. 7B).

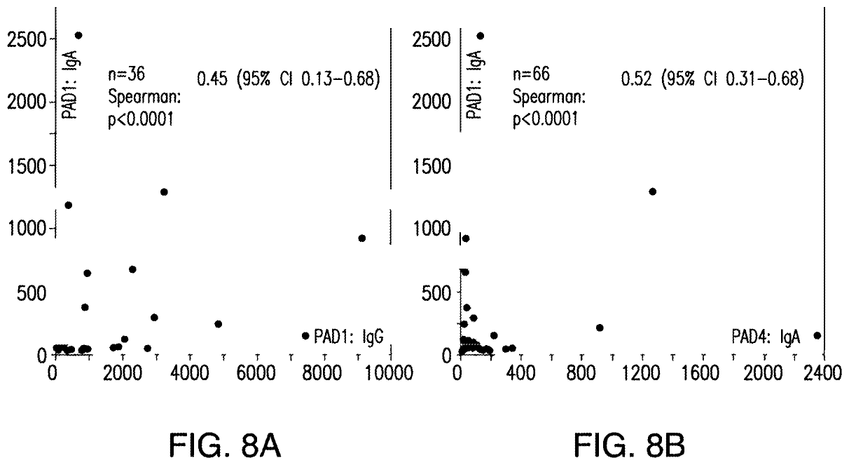

Comparison between the levels of anti-PAD1 IgG and anti-PAD1 IgA was also performed. The results indicated that although a significant correlation was observed, individual patients had varying levels of anti-PAD1 IgA and anti-PAD1 IgG to PAD1 (FIG. 8A). A correlation between anti-PAD1 IgA and anti-PAD4 IgA was also performed, and also revealed a significant correlation (FIG. 8B) between the IgA isotypes for anti-PAD1 and anti-PAD4. Surprisingly, several patients were highly positive on anti-PAD1 IgA but negative on anti-PAD4 IgA, which indicated that anti-PAD1 IgA could identify some patients with RA that may be negative for anti-PAD4.

Taken together these results demonstrate that anti-PAD1 IgA is able to discriminate RA from controls, and can be used alone or in combination with detection of the other autoantibodies against PAD autoantigens.

### Example VI

### IgG, IgA, and IgM Isotypes of Anti-PAD4 Identified in Sera of RA Patients

This example demonstrates that anti-PAD4 with IgG, IgA, and IgM isotypes can be detected in RA patients.

To further evaluate which isotypes of anti-PAD4 could be detected in RA patients, PAD4-coupled beads were tested with anti-human IgM, IgA and IgG conjugates on an extended cohort of RA patients (n=62) and the same non-RA controls from Cohort I (n=36).

The results for the extended testing of anti-PAD4 with IgG, IgA and IgM, revealed that all three isotypes were identified in the sera of RA patients. Higher levels of the three isotypes were observed in RA patients with erosive disease when compared with the patients without erosion, but this association was only significant for anti-PAD4 IgA (p=0.0086).

### Example VII

### Anti-PAD1 Antibodies Recognized Different Epitope than Anti-Citrullinated Protein/Peptide Antibodies

This example demonstrates that the anti-PAD1 antibodies detected in RA patients recognize unique epitopes in the PAD1 enzyme that are distinct from the citrullinated epitopes recognized by anti-citrullinated protein/peptide antibodies (ACPA).

### Objectives

The objective of this study was to characterize the PAD proteins used in the assays as targets of the anti-PAD1 antibodies, and in particular, to analyze their potential autocitrullination and citrullination status.

### Methods

### Native and Citrullinated PAD Antigens Generation

To generate native and citrullinated PAD1 antigens, the full-length PAD1 protein was recombinantly expressed in bacterial cells and purified by propietary chromatographic techniques in the absence of calcium (the enzyme's cofactor) or with 5-10 mM CaCl₂) present in the extraction and purification buffers.

### Citrullination Status Analysis

Citrullination status of the different PAD antigens was confirmed by immunoblotting with the anti-Citrulline (Modified) Detection Kit (EMD Millipore, Cat. #17-347B).

Preparation of the Positive Control (In Vitro Citrullinated Histone)

H3.1 human recombinant histone (New England Biolabs. P/N:M2503S) was incubated with 8.571 U/mg of a commercial human recombinant PAD4 in a buffer containing 100 mM HEPES pH 7.6, 10 mM CaCl₂), 5 mM DTT for 2.5 hours at 37° C.

Anti-Modified Citrulline (AMC) Immunoblotting

The following antigens were tested with the AMC assay:

| | | | Presence of | Expected |
|---|---|---|---|---|
| | | | calcium during | citrullination |
| Antigen | Source | Lot # | its generation | status |
| PAD1 | In-house generated | 1 | Yes | Yes |
| PAD1 | In-house generated | 2 | No | No |
| PAD1 | In-house generated | 3 | Yes | Yes |
| PAD1 | In-house generated | 4 | No | No |
| PAD1 | In-house generated | 5 | No | No |
| PAD1 | Commercial | 1 | Not disclosed | No |
| PAD4 | Commercial | 1 | Not disclosed | No |
| PAD6 | In-house generated | 1 | No | No |
| PAD6 | Commercial | 1 | Not disclosed | No |
| Streptolysin O (SLO) | In-house generated | — | Non-citrullinated | No |
| (negative control 1) | | | | |
| Histone H3.1 | New England Biolabs | 0041312 | Non-citrullinated | No |
| (negative control 2) | Cat. #M2503S | | | |
| Histone H3.1; 2 μg/well | New England Biolabs | 0041312 | In vitro | Yes |
| (positive control 1) | Cat. #M2503S | | citrullinated | |
| Histone H3.1; 4 μg/well | New England Biolabs | 0041312 | In vitro | Yes |
| (positive control 2) | Cat. #M2503S | | citrullinated | |

The assay was run following the manufacturer's procedure. In short, SDS-polyacrylamide gel electrophoresis (SDS-PAGE) was performed with 2 μg/well of the different protein (citrullinated histone positive control was run at 2 and 4 μg/well) samples and the proteins from one of the gels was transferred to a PVDF membrane. The modification buffer was prepared following the manufacturing instructions and it was added to the blot. The blow was placed in a light-proof container and incubated at 37° C. overnight without agitation. The blot was then rinsed with water and the blot was blocked with 5% non-fat dry milk in TBS-Tween for 1 hour. After that, the blot was incubated with 10 mL of a 1:1000 dilution of anti-Modified Citrulline antibody diluted in freshly prepared TBST-MILK for 2 hours at room temperature with constant agitation. After washing, the blot was incubated with 10 mL of 1:2000 dilution of the goat a-human HRP Conjugate in 1% milk in TBS-Tween for 1 hour at room temperature with constant agitation. The membrane was washed again, developed with SuperSignal West Pico PLUS Chemiluminescent Substrate, and read with the iBright FL1000 Imaging System.

Aptiva Assays and RA Sera Testing

A panel for the detection of anti-PAD1 IgG based on a particle-based multi-analyte technology [PMAT, research use only (RUO), Inova Diagnostics, San Diego, US] was created as previously described using the different PAD1 antigen versions, including a commercial PAD1, three in-house PAD1 antigens produced without Ca²⁺ (Lot #2, Lot #4, and Lot #5), and two in-house PAD1 antigens produced with Ca²⁺ (Lot #1, and Lot #3). The testing reaction was performed on a research instrument based on the Aptiva® technology (Inova Diagnostics, San Diego, US, RUO). The anti-PAD1 IgG panel was used to test sera from RA patients (n=22) of expected different anti-PAD1 status based on a commercial PAD1 antigen.

### Results

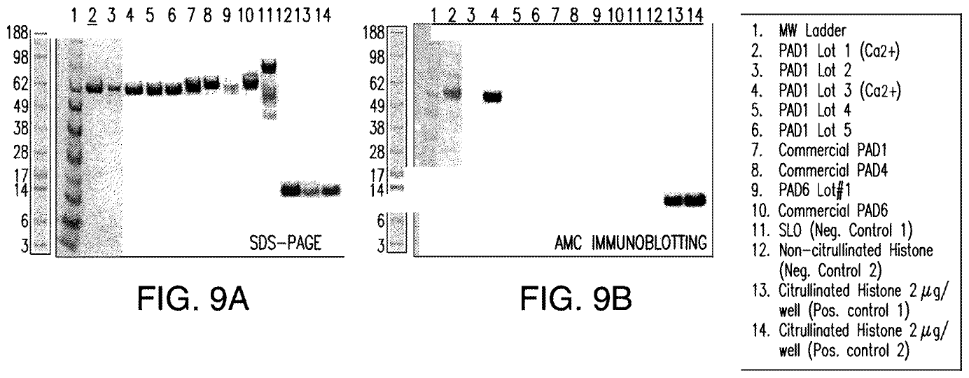

A strong and defined band was observed in all lanes at the expected molecular weight for each protein in the SDS-PAGE gel (FIG. 9A).

In the anti-modified citrulline (AMC) blot (FIG. 9B), no bands were observed for either of the negative controls (lanes 11 and 12). Strong and proportional bands were observed for the positive control at the two concentrations tested (lanes 13 and 14). As expected, only the PAD1 antigens generated in the presence of calcium (lanes 2 and 4) showed a band in the blot, indicating that these proteins are citrullinated and that therefore, they had undergone autocitrullination during their generation. No bands could be observed in the blot for the PAD1 antigens generated in the absence of calcium or for the PAD4 or PAD6 antigens included, indicating that these proteins are not citrullinated. Interestingly, a weak band could be observed for the commercial PAD1 protein, which indicated partial citrullination.

### Conclusions

Taken together, the results indicated that the anti-PAD1 antibodies recognize non-citrullinated PAD1, and are distinct from ACPA.

### EMBODIMENTS

1. A method of diagnosing rheumatoid arthritis (RA), comprising:
(a) contacting a biological sample from a subject suspected of having RA with at least one peptidyl arginine deiminase (PAD) protein or an antigenic fragment thereof, and
(b) detecting the presence of an autoantibody reactive with the at least one PAD protein or an antigenic fragment thereof, wherein the presence of said autoantibody is indicative of RA,
wherein the at least one PAD protein comprises PAD1, or PAD1 and PAD4.

2. The method of embodiment 1, wherein the at least one PAD protein is PAD1 or an antigenic fragment thereof.

3. The method of embodiment 1, wherein the at least one PAD protein is PAD1 and PAD4 or an antigenic fragment thereof.

4. The method of any one of embodiments 1 to 3, wherein the at least one PAD protein further comprises one or more PAD protein selected from the group consisting of PAD2, PAD3, and PAD6 or an antigenic fragment thereof.

5. The method of embodiment 4, wherein the at least one PAD protein is PAD1, PAD4, and PAD2 or an antigenic fragment thereof.

6. The method of embodiment 4, wherein the at least one PAD protein is PAD1, PAD4, and PAD3 or an antigenic fragment thereof.

7. The method of embodiment 4, wherein the at least one PAD protein is PAD1, PAD4, PAD2, and PAD3 or an antigenic fragment thereof.

8. The method of embodiment 4, wherein the at least one PAD protein is PAD1, PAD4, PAD2, PAD3, and PAD6 or an antigenic fragment thereof.

9. A method of monitoring the progression of rheumatoid arthritis (RA), comprising:
(a) contacting a biological sample from a subject having or suspected of having RA with at least one peptidyl arginine deiminase (PAD) protein or an antigenic fragment thereof, and
(b) detecting the presence of an autoantibody reactive with the at least one PAD protein or an antigenic fragment thereof, wherein the presence of said autoantibody is indicative of disease progression,
wherein the at least one PAD protein comprises PAD1, or PAD1 and PAD4.

10. The method of embodiment 9, wherein the at least one PAD protein is PAD1 or an antigenic fragment thereof.

11. The method of embodiment 9, wherein the at least one PAD protein is PAD1 and PAD4 or an antigenic fragment thereof.

12. The method of any one of embodiments 9 to 11, wherein the at least one PAD protein further comprises PAD3 or an antigenic fragment thereof.

13. The method of any one of embodiments 9 to 12, wherein the presence of said autoantibody is indicative of RA stage.

14. A method of monitoring the progression of rheumatoid arthritis (RA), comprising:
(a) contacting a biological sample from a subject having RA with at least one peptidyl arginine deiminase (PAD) protein or an antigenic fragment thereof, and
(b) detecting the absence of an autoantibody bound to the at least one PAD protein or an antigenic fragment thereof, wherein the absence of said autoantibody is indicative of disease progression,
wherein the at least one PAD protein comprises PAD1, or PAD1 and PAD4.

15. The method of embodiment 14, wherein the at least one PAD protein is PAD1 or an antigenic fragment thereof.

16. The method of embodiment 14, wherein the at least one PAD protein is PAD1 and PAD4 or an antigenic fragment thereof.

17. The method of any one of embodiments 14 to 16, wherein the at least one PAD protein further comprises PAD3 or an antigenic fragment thereof.

18. The method of any one of embodiments 14 to 17, wherein the absence of said autoantibody is indicative of RA stage.

19. The method of any one of embodiments 1 to 18, wherein said biological sample comprises whole blood, serum, plasma synovial fluid or sputum.

20. The method of any one of embodiments 1 to 19, wherein said biological sample comprises serum or plasma.

21. The method of any one of embodiments 1 to 20, wherein said antigenic fragment comprises from 6-120, 12-100, 18-80, 24-60, 30-50 or 35-45 amino acid residues.

22. The method of any one of embodiments 1 to 21, wherein said PAD protein or antigenic fragment thereof is obtained by a method comprising isolation from a natural source, chemical synthesis or recombinant expression.

23. The method of any one of embodiments 1 to 22, wherein said PAD protein or antigenic fragment thereof is obtained by chemical synthesis.

24. The method of any one of embodiments 1 to 23, wherein said detection comprises an immunoassay.

25. The method of embodiment 24, wherein said immunoassay is selected from the group consisting of a fluorescent immunosorbent assay (FIA), a chemiluminescent immunoassay (CIA), a radioimmunoassay (RIA), multiplex immunoassay, a protein/peptide array immunoassay, a solid phase radioimmunoassay (SPRIA), an indirect immunofluorescence assay (IIF), an enzyme linked immunosorbent assay (ELISA), a particle based multianalyte test (PMAT), and a Dot Blot assay.

26. The method of any one of embodiments 1 to 25, wherein said detection comprises contacting said autoantibody bound to the PAD protein or antigenic fragment thereof with a detection probe.

27. The method of embodiment 26, wherein said detection probe binds to said autoantibody.

28. The method of embodiment 26 or 27, wherein said detection probe comprises an antibody or functional fragment thereof.

29. The method of embodiment 26 or 27, wherein said detection probe comprises a reporter tag.

30. The method of embodiment 29, wherein said reporter tag is a label.

31. The method of embodiment 30, wherein said label is selected from the group consisting of a fluorophore, enzyme, chemiluminescent moiety, radioactive moiety, organic dye and small molecule.

32. The method of embodiment 30 or 31, wherein said label is a fluorescent label.

33. The method of embodiment 32, wherein said fluorescent label is phycoerytherin (PE).

34. The method of embodiment 29, wherein said reporter tag comprises a ligand or a particle.

35. The method of embodiment 34, wherein said ligand is biotin.

36. The method of embodiment 34, wherein said particle comprises a nanoparticle.

37. A detection kit, comprising:
at least one peptidyl arginine deiminase (PAD) protein, or an antigenic fragment thereof, that can capture an autoantibody specific to the PAD protein;
a detection probe that recognizes said autoantibody, and
a solid support,
wherein the at least one PAD protein comprises PAD1, or PAD1 and PAD4.

38. The kit of embodiment 37, wherein the at least one PAD protein is PAD1 or an antigenic fragment thereof.

39. The kit of embodiment 37, wherein the at least one PAD protein is PAD1 and PAD4 or an antigenic fragment thereof.

40. The kit of any one of embodiments 37 to 39, wherein the at least one PAD protein further comprises one or more PAD protein selected from the group consisting of PAD2, PAD3, and PAD6 or an antigenic fragment thereof.

41. The kit of embodiment 40, wherein the at least one PAD protein is PAD1, PAD4, and PAD2 or an antigenic fragment thereof.

42. The kit of embodiment 40, wherein the at least one PAD protein is PAD1, PAD4, and PAD3 or an antigenic fragment thereof.

43. The kit of embodiment 40, wherein the at least one PAD protein is PAD1, PAD4, PAD2, and PAD3 or an antigenic fragment thereof.

44. The kit of embodiment 40, wherein the at least one PAD protein is PAD1, PAD4, PAD2, PAD3, and PAD6 or an antigenic fragment thereof.

45. The kit of any one of embodiments 37 to 44, further comprising a label.

46. The kit of embodiment 45, wherein said label is selected from the group consisting of a fluorophore, enzyme, chemiluminescent moiety, radioactive moiety, organic dye and small molecule.

47. The kit of any one of embodiments 37 to 46, further comprising a positive control.

48. The kit of any one of embodiments 37 to 47, further comprising one or more ancillary reagents.

49. The kit of embodiment 48, wherein said one or more ancillary reagents is selected from the group consisting of an incubation buffer, a wash buffer, a detection buffer and a detection instrument.

50. The kit of any one of embodiments 37 to 49, wherein said antigenic fragment comprises from 6-120, 12-100, 18-80, 24-60, 30-50 or 35-45 amino acid residues.

51. The kit of any one of embodiments 37 to 50, wherein said detection probe comprises an antibody or functional fragment thereof.

52. The kit of any one of embodiments 37 to 50, wherein said detection probe comprises a reporter tag.

53. The kit of embodiment 52, wherein said reporter tag is a label.

54. The kit of embodiment 45 or 53, wherein said label is selected from the group consisting of a fluorophore, enzyme, chemiluminescent moiety, radioactive moiety, organic dye and small molecule.

55. The kit of embodiment 45, 53, or 54, wherein said label is a fluorescent label.

56. The kit of embodiment 55, wherein said fluorescent label is phycoerytherin (PE).

57. The kit of embodiment 53, wherein said reporter tag comprises a ligand or particle.

58. The kit of embodiment 57, wherein said ligand is biotin.

59. The kit of embodiment 57, wherein said particle comprises a nanoparticle.

60. The kit of any one of embodiments 37 to 59, wherein said solid support is selected from the group consisting of a bead, sphere, particle, membrane, chip, slide, plate, well and test tube.

61. The kit of embodiment 60, wherein said bead, sphere or particle has a diameter of about 0.1 to about 100 micrometer.

62. The kit of embodiment 60, wherein said membrane is selected from the group consisting of nitrocellulose, nylon, polyvinylidene fluoride (PVDF) and polyvinylidene difluoride.

63. The kit of any one of embodiments 37 to 62, wherein said PAD protein or antigenic fragment thereof is conjugated to said solid support.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of diagnosing rheumatoid arthritis (RA), comprising:
(a) contacting a biological sample from a subject suspected of having RA with at least one peptidyl arginine deiminase (PAD) protein or an antigenic fragment thereof, and
(b) detecting the presence of an autoantibody reactive with the at least one PAD protein or an antigenic fragment thereof, wherein the presence of said autoantibody is indicative of RA,
wherein the at least one PAD protein comprises PAD1, or PAD1 and PAD4, and optionally further comprises one or more PAD protein selected from the group consisting of PAD2, PAD3, and PAD6 or an antigenic fragment thereof.

2. The method of claim 1, wherein the at least one PAD protein comprises
(a) PAD1 or an antigenic fragment thereof,
(b) PAD1 and PAD4 or an antigenic fragment thereof,
(c) PAD1, PAD4, and PAD2 or an antigenic fragment thereof,
(d) PAD1, PAD4, and PAD3 or an antigenic fragment thereof,
(e) PAD1, PAD4, PAD2, and PAD3 or an antigenic fragment thereof, or
(f) PAD1, PAD4, PAD2, PAD3, and PAD6 or an antigenic fragment thereof.

3. The method of claim 1, wherein said biological sample comprises
(a) whole blood, serum, plasma synovial fluid or sputum; or
(b) serum or plasma.

4. The method of claim 1, wherein said antigenic fragment comprises from 6-120, 12-100, 18-80, 24-60, 30-50 or 35-45 amino acid residues.

5. The method of claim 1, wherein said PAD protein or antigenic fragment thereof is obtained by
(a) a method comprising isolation from a natural source, chemical synthesis or recombinant expression; or
(b) a method comprising chemical synthesis.

6. The method of claim 1, wherein said detection comprises:
(a) an immunoassay,
wherein said immunoassay is optionally selected from the group consisting of a fluorescent immunosorbent assay (FIA), a chemiluminescent immunoassay (CIA), a radioimmunoassay (RIA), multiplex immunoassay, a protein/peptide array immunoassay, a solid phase radioimmunoassay (SPRIA), an indirect immunofluorescence assay (IIF), an enzyme linked immunosorbent assay (ELISA), a particle based multianalyte test (PMAT), and a Dot Blot assay; and/or
(b) contacting said autoantibody bound to the PAD protein or antigenic fragment thereof with a detection probe,
wherein said detection probe optionally:
(i) binds to said autoantibody;
(ii) comprises an antibody or functional fragment thereof, and/or
(iii) comprises a reporter tag,
wherein said reporter tag optionally comprises a label, wherein the label optionally is selected from the group consisting of a fluorophore, enzyme, chemiluminescent moiety, radioactive moiety, organic dye and small molecule, and/or optionally is a fluorescent label, and
wherein said fluorescent label is optionally phycoerytherin (PE); or
(iv) a ligand or a particle, and wherein said ligand optionally comprises biotin or a nanoparticle.

7. A method of monitoring the progression of rheumatoid arthritis (RA), comprising:
(a) contacting a biological sample from a subject having or suspected of having RA with at least one peptidyl arginine deiminase (PAD) protein or an antigenic fragment thereof, and
(b) detecting:
(i) a presence of an autoantibody reactive with the at least one PAD protein or an antigenic fragment thereof, wherein the presence of said autoantibody is indicative of disease progression, and/or
(ii) an absence of an autoantibody bound to the at least one PAD protein or an antigenic fragment thereof, wherein the absence of said autoantibody is indicative of disease progression,
wherein the at least one PAD protein comprises PAD1, or PAD1 and PAD4, and optionally further comprises PAD3 or an antigenic fragment thereof.

8. The method of claim 7, wherein the at least one PAD protein comprises:
(a) PAD1 or an antigenic fragment thereof;
(b) PAD1 and PAD4 or an antigenic fragment thereof

9. The method of claim 7, wherein the presence of said autoantibody is indicative of RA stage.

10. The method of claim 7, wherein said biological sample comprises
(a) whole blood, serum, plasma synovial fluid or sputum; or
(b) serum or plasma.

11. The method of claim 7, wherein said antigenic fragment comprises from 6-120, 12-100, 18-80, 24-60, 30-50 or 35-45 amino acid residues.

12. The method of claim 7, wherein said PAD protein or antigenic fragment thereof is obtained by:
(a) a method comprising isolation from a natural source, chemical synthesis or recombinant expression; or
(b) a method comprising chemical synthesis.

13. The method of claim 7, wherein said detection comprises:
(a) an immunoassay,
wherein said immunoassay is optionally selected from the group consisting of a fluorescent immunosorbent assay (FIA), a chemiluminescent immunoassay (CIA), a radioimmunoassay (RIA), multiplex immunoassay, a protein/peptide array immunoassay, a solid phase radioimmunoassay (SPRIA), an indirect immunofluorescence assay (IIF), an enzyme linked immunosorbent assay (ELISA), a particle based multianalyte test (PMAT), and a Dot Blot assay; and/or
(b) contacting said autoantibody bound to the PAD protein or antigenic fragment thereof with a detection probe,
wherein said detection probe optionally:
(i) binds to said autoantibody;
(ii) comprises an antibody or functional fragment thereof, and/or
(iii) comprises a reporter tag,
wherein said reporter tag optionally comprises a label, wherein the label optionally is selected from the group consisting of a fluorophore, enzyme, chemiluminescent moiety, radioactive moiety, organic dye and small molecule, and/or optionally is a fluorescent label, and
wherein said fluorescent label is optionally phycoerytherin (PE); or
(iv) a ligand or a particle, and wherein said ligand optionally comprises biotin or a nanoparticle.

14. A detection kit, comprising:
(a) at least one peptidyl arginine deiminase (PAD) protein, or an antigenic fragment thereof, that can capture an autoantibody specific to the PAD protein;
(b) a detection probe that recognizes said autoantibody, and
(c) a solid support,
wherein the at least one PAD protein comprises PAD1, or PAD1 and PAD4, and optionally further comprises one or more PAD protein selected from the group consisting of PAD2, PAD3, and PAD6 or an antigenic fragment thereof.

15. The kit of claim 14, wherein the at least one PAD protein is:
(a) PAD1 or an antigenic fragment thereof
(b) PAD1 and PAD4 or an antigenic fragment thereof,
(c) PAD1, PAD4, and PAD2 or an antigenic fragment thereof,
(d) PAD1, PAD4, PAD2, and PAD3 or an antigenic fragment thereof,
(e) PAD1, PAD4, PAD2, and PAD3 or an antigenic fragment thereof,
(f) PAD1, PAD4, PAD2, PAD3, and PAD6 or an antigenic fragment thereof

16. The kit of claim 14, further comprising:
(a) a label, wherein said label is optionally selected from the group consisting of a fluorophore, enzyme, chemiluminescent moiety, radioactive moiety, organic dye and small molecule;
(b) a positive control;
(c) one or more ancillary reagents, wherein said one or more ancillary reagents is optionally selected from the group consisting of an incubation buffer, a wash buffer, a detection buffer and a detection instrument

17. The kit of claim 14, wherein said antigenic fragment comprises from 6-120, 12-100, 18-80, 24-60, 30-50 or 35-45 amino acid residues.

18. The kit of claim 14, wherein said detection probe:
(a) binds to said autoantibody;
(b) comprises an antibody or functional fragment thereof, and/or
(c) comprises a reporter tag, wherein said reporter tag optionally comprises:
(i) a label, wherein the label optionally is selected from the group consisting of a fluorophore, enzyme, chemiluminescent moiety, radioactive moiety, organic dye and small molecule, and/or optionally is a fluorescent label, wherein
wherein said fluorescent label is optionally phycoerytherin (PE); or
(ii) a ligand or a particle, and wherein said ligand optionally comprises biotin or a nanoparticle.

19. The kit of claim 14, wherein said solid support is selected from the group consisting of a bead, sphere, particle, membrane, chip, slide, plate, well and test tube,
wherein said bead, sphere or particle optionally has a diameter of about 0.1 to about 100 micrometer; and
wherein said membrane is optionally selected from the group consisting of nitrocellulose, nylon, polyvinylidene fluoride (PVDF) and polyvinylidene difluoride.

20. The kit of claim 14, wherein said PAD protein or antigenic fragment thereof is conjugated to said solid support.
